(19) United States
(12) Patent Application Publication
(10) Pub. No.: US 2025/0360060 A1
(43) Pub. Date: Nov. 27, 2025

(54) BODY LOTION COMPOSITION WITH LAMELLAR GEL NETWORK

(71) Applicant: The Procter & Gamble Company, Cincinnati, OH (US)

(72) Inventors: Colin John Clarke, Basingstoke (GB); Shawn David McConaughy, Cincinnati, OH (US); Kelly Rose Kroger Lyons, Liberty Township, OH (US)

(21) Appl. No.: 19/215,997

(22) Filed: May 22, 2025

Related U.S. Application Data
(60) Provisional application No. 63/651,220, filed on May 23, 2024.

(51) Int. Cl. A61K 8/04; A61K 8/55; A61Q 19/00

(52) U.S. Cl. CPC A61K 8/042; A61K 8/55; A61Q 19/007; A61K 2800/30; A61K 2800/596

(57) ABSTRACT

A skin care composition that includes an emulsifier, a fatty amphiphile, a humectant, an emollient, and an aqueous carrier. The emulsifier includes an anionic surfactant and a non-ionic surfactant and at least a portion of the anionic surfactant, non-ionic surfactant, and fatty amphiphile form a lamellar gel network structure. The skin care composition can optionally include a polymer, such as an Caesalpinia spinosa Gum/Ammonium AMPS Crosspolymer.

### FIELD OF THE INVENTION

The present disclosure generally relates to a body lotion composition. More particularly, to body lotion compositions having a lamellar gel network that helps provide improved skin feel.

### BACKGROUND OF THE INVENTION

Body lotion is a widely used cosmetic product designed to provide hydration and nourishment to the skin. Its purpose is to maintain the skin's moisture balance, enhance its texture, and promote overall skin health. Many consumers apply body lotions after bathing or showering because water and cleansing products can strip away natural oils and moisture from the skin, leaving skin feeling dry, tight, and dehydrated.

However, conventional body lotions frequently impart an undesirable greasy or sticky residue on the skin, primarily due to the high concentrations of emollients or humectants utilized in their formulation. This can lead to a prolonged period for absorption, resulting in a wet and/or heavy sensation for formulations rich in humectants, and a greasy feel for products with high emollient content. Moreover, some consumers may find the need for frequent reapplication throughout the day to maintain optimal skin hydration and nourishment. Additionally, if consumers apply clothing or get into bed before the body lotion is fully absorbed, there is a risk of transfer onto their garments and sheets, which can be inconvenient and potentially stain the fabric.

Furthermore, the problems of greasiness, stickiness, and slow absorption are intensified when consumers apply body lotion immediately after bathing or showering. This is because the skin rapidly loses the excess moisture obtained during bathing or showering, and as the moisture evaporates from the skin, it hinders the absorption of the lotion. Consequently, this prolonged adsorption time exacerbates issues of greasiness and stickiness, and makes the skin feel oilier and tackier.

Therefore, there is a need for a body lotion composition that includes a high level of emollients and/or humectants without feeling sticky, heavy, and/or greasy.

### SUMMARY OF THE INVENTION

A skin care composition comprising: (a) an emulsifier comprising an anionic surfactant chosen from an alkyl phosphate monoester, an alkyl phosphate diester, salts thereof, or mixtures thereof; and a non-ionic surfactant; (b) a fatty amphiphile; (c) from about 0.1% to about 20%, of a humectant; (d) from about 0.1% to about 20% of an emollient; an aqueous carrier; wherein at least a portion of the anionic surfactant, non-ionic surfactant, and fatty amphiphile form a lamellar gel network structure.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1A shows Example 4 at 700× magnification;

FIG. 1B shows Example 4 diluted with water by 50% at 700× magnification;

FIG. 1C shows Example 1 at 700× magnification;

FIG. 2A shows Example 4 at 700× magnification, providing a different image of the same example presented in FIG. 1A;

FIG. 2B shows CeraVe® Moisturizing Face & Body Cream for Normal to Dry Skin at 700× magnification;

FIG. 2C shows CeraVe® Daily Moisturizing Face and Body Lotion for Normal to Dry Skin at 700× magnification;

FIG. 2D shows Aveeno® Daily Moisturizing Cream at 700× magnification;

FIG. 2E shows Naturium® Bio-Lipid Restoring Body Lotion at 700× magnification;

FIG. 2F shows Example 1 at 700× magnification, this image is identical to FIGS. 1C and 1s included again as FIG. 2F to facilitate easier comparison with the other images in FIG. 2;

FIG. 2G shows Example 4 with NaCl solution at 700× magnification;

FIG. 2H shows CeraVe® Moisturizing Face & Body Cream for Normal to Dry Skin with NaCl solution at 700× magnification;

FIG. 2I shows CeraVe® Daily Moisturizing Face and Body Lotion for Normal to Dry Skin with NaCl solution at 700× magnification;

FIG. 2J shows Aveeno® Daily Moisturizing Cream with NaCl solution at 700× magnification;

FIG. 2K shows Naturium® Bio-Lipid Restoring Body Lotion with NaCl solution at 700× magnification;

FIG. 2L shows Example 1 with NaCl solution at 700× magnification;

FIG. 3 shows the viscosity profile (viscosity vs. s⁻¹) of Example 4 when it is neat, diluted with 10% water, diluted with water, and diluted with 50% water;

FIG. 4 is a graphic display of the curve created by the DSC method for Example 4;

FIGS. 5A and 5B show cryo-SEM images of a sample of Example 4;

FIG. 6 shows a cryo-SEM image of a sample of Olay® Firming & Hydrating Body Lotion with Collagen; and

FIG. 7 illustrates a setup for the Tack Method.

### DETAILED DESCRIPTION OF THE INVENTION

Current body lotion products provide skin hydration and nourishment by incorporating either emollients or humectants but seldom use high levels of both ingredients. Unfortunately, current products often suffer from a perceived slow absorption rate and can leave a greasy and/or tacky residue on the skin. As a result, many consumers have adapted their routine to wait several minutes after application before getting dressed, putting on pajamas, or getting into bed. Furthermore, due to its structure, body lotion is not tolerant of high salt levels. This can affect the concentration of active ingredients, many of which are salts themselves. Consumers often report that when they sweat or swim in a pool or the ocean while wearing body lotion, the lotion can change texture. As a result, it may feel greasy or slimy on the skin. To address these limitations, there is a need for a body lotion formulation that combines a relatively high level of emollients and/or humectants to deliver superior performance without the drawbacks of a heavy, greasy, and/or tacky feel.

It was surprisingly found that a lotion composition that included a lamellar gel network phase, a relatively high level of emollients (e.g., ≥5%, ≥7%) and/or humectants (e.g., ≥5%, ≥7%), and an aqueous carrier can have improved skin feel and be more tolerant to salt. The lamellar gel network can include an anionic surfactant, in particular a mono and di-cetyl phosphate ester, a fatty amphiphile such as monohydric fatty amphiphile, and a non-ionic co-surfactant.

It was found that the lamellar gel network leverages the repulsion of the anionic surfactant resulting in an optimized d-spacing. This structural arrangement allows the composition to retain a higher amount of water internally, enabling higher levels of humectants and/or emollients. As a result, the composition delivers a perception of fast absorption without leaving a greasy or tacky residue. Many current body lotion products include a micellar structure with polymeric thickeners and the emollient coalesces to form larger droplets, which can feel oily, sticky, and/or slow to absorb. The lamellar gel network structure helps to disperse the emollient droplets into the composition at small particle sizes. 50% or more of the emollient droplets can have a diameter of from about 1 μm to about 20 μm, from about 1 μm to about 10 μm, from about 1 to about 5 μm, and from about 2 μm to about 4 μm. 90% of the emollient droplets can have a diameter of less than 20 μm, less than or equal to 15 μm, less than or equal to 10 μm. The droplet size can be measured as follows: a small droplet of the composition is applied to a microscope slide with a micro pipette. A cover glass is applied and gently pressed. The slides are presented to a Keyence VHX 7000 Microscope and Images are captured using VHX Image capture software. Particle size can be evaluated by the addition of a 10 μm calibrated scale to the image from the software.

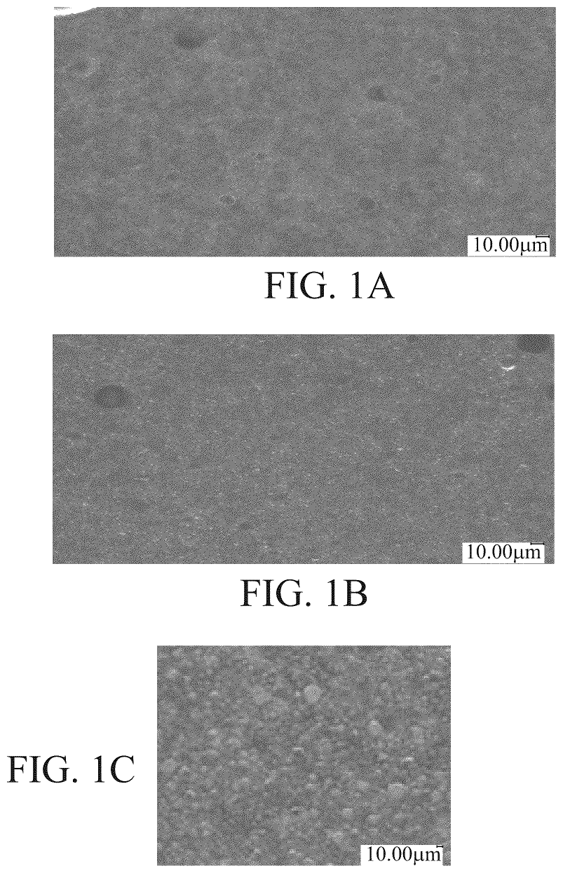

FIG. 1A shows Example 4 (Table 7, hereafter), a lotion example with a lamellar gel network, at 700× magnification and FIG. 1B shows Example 4 diluted with water by 50% at 700× magnification. Both FIGS. 1A and 1B show small, bright droplets that represent the dispersed emollient droplets within the composition. Notably, both figures show these emollient droplets and both have a similar pattern that indicates the underlying structure remains intact, even when the composition is diluted by 50%.

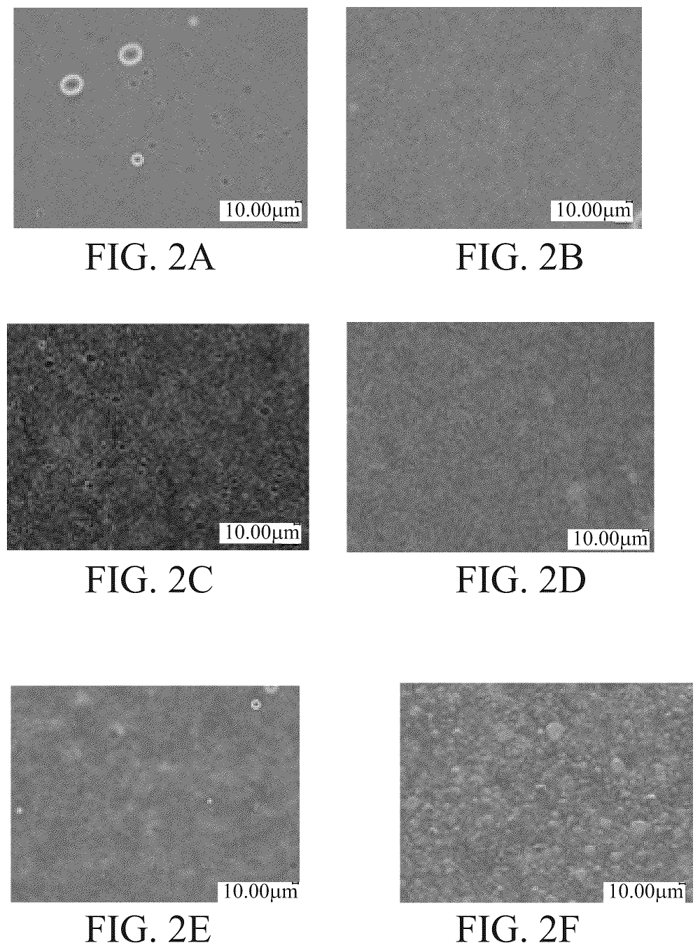

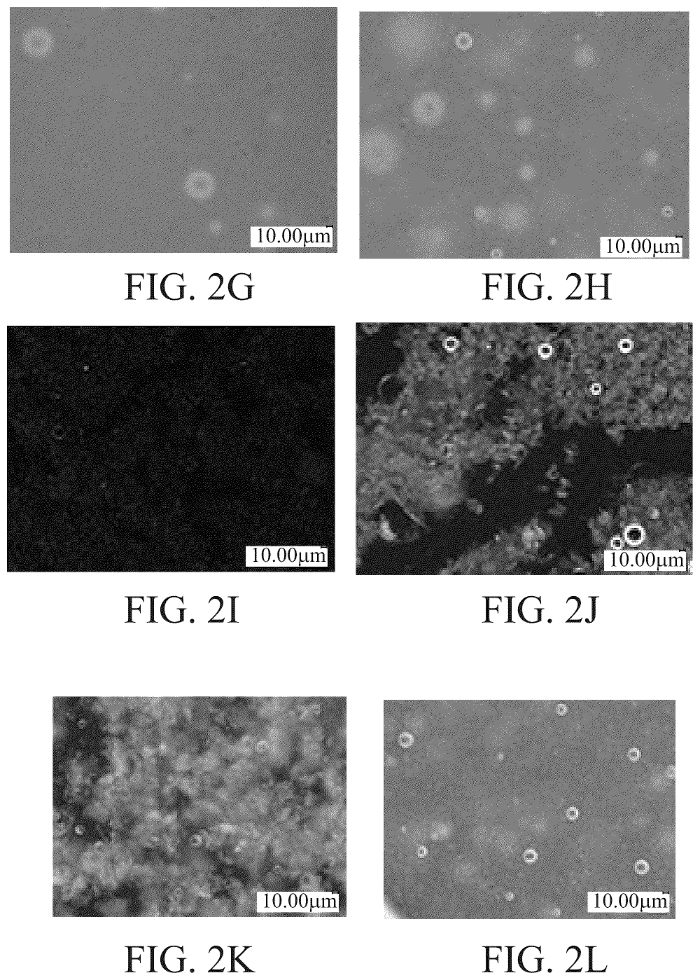

FIG. 2F shows Example 2F at 700X, which has a micellar structure. As shown in FIG. 2F, the emollient droplets are significantly larger than the oil droplets shown in FIGS. 1A and 1B and many are >20 μm. In Example 2F, there is no gel network structure, and the emollient tends to coalesce to form these larger droplets.

It was found that the addition of the non-ionic co-surfactant, as in Example 4, helped facilitate formation of a lamellar gel network and lotion compositions with this lamellar gel network was found to significantly improve the performance of the lotion, especially when it was diluted or applied to wet or damp skin or in high humidity conditions. This outcome was particularly notable because it addressed a common problem observed in many existing moisturizers. These products often encounter issues such as separation, curdling, sliminess, difficulties in achieving even spreadability, and/or difficulties adsorbing into skin when applied to wet or damp skin, which can arise after showering or bathing, before the user has completely dried off, or due to the skin rapidly losing excess moisture post-shower or bath.

The lamellar gel network structure offers notable advantages. First, it can effectively trap water within its bilayers, allowing the lotion to be applied effectively on wet or damp skin. The gel network structure absorbs water through dilution of anionic surfactants charge, which causes the anionic charges to push further away from each layer in the gel network. This allows the non-ionic surfactant's headgroup to further expand and absorb some of the additional water thereby increasing the d-layer spacing of the lamellar structure. This enables the composition to better maintain its structure and skin properties when applied, even if it is applied to wet skin or otherwise diluted or applied in high humidity conditions.

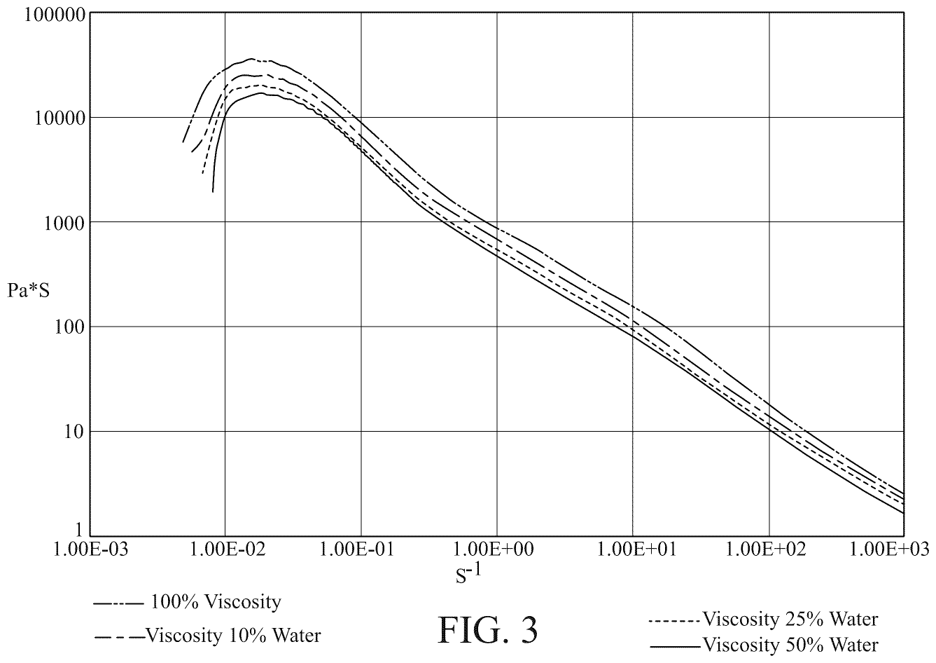

FIG. 3 and Table 2 show the peak viscosity when an example is neat and when the example is diluted with 10%, 25%, and 50% water and mixed. The viscosity and peak viscosity was measured according to the Viscosity Test Method, described hereafter.

FIG. 3 shows the viscosity profile (viscosity vs. shear rate) of Example 4 (Table 7, hereafter) when it is neat, diluted with 10% water, diluted with 25% water, and diluted with 50% water. As shown in FIG. 3, the curves have a similar shape, which indicates that the viscosity, including the peak viscosity, does not significantly decline when water is added to the example.

Table 2 compares the peak viscosity of Examples 4 (Table 7, hereafter) and the following commercially available products: Olay® Firming & Hydrating Body Lotion with Collagen, Nivea® Breathable Nourishing Body Lotion and Jergens® Ultra Healing Hand and Body Lotion. The list of ingredients for the commercial products is in Table 1, below. In Table 2, the retention of viscosity at 25% dilution was calculated by dividing the peak viscosity at 25% dilution by the peak viscosity of the neat sample. Similarly, the retention of viscosity at 50% dilution was calculated by dividing the peak viscosity at 50% dilution and by the peak viscosity in the neat sample.

Table 2 shows that Example 4, which had a lamellar gel network structure, had superior viscosity retention when diluted by 10%, 25%, and 50%, as compared to Example 4 (without significant lamellar gel network structure) and the commercial examples. This suggests that when applied to the skin, Example 4 may feel lighter and less sticky, especially in situations where the skin is wet or damp, or when the user has recently taken a shower or bath.

The neat composition can have a peak viscosity of from about 1500 Pa*s to about 50,000 Pa*s, from about 5000 Pa*s to about 45,000 Pa*s, from about 7500 Pa*s to about 40,000 Pa*s, from about 15,000 Pa*s to about 40,000 Pa*s, from about 20,000 Pa*s to about 37,000 Pa*s according to the Viscosity Test Method.

The composition can have a peak viscosity at 25% dilution of from about 1200 Pa*s to about 35,000 Pa*s, from about 5000 Pa*s to about 25,000 Pa*s, from about 7500 Pa*s to about 25,000 Pa*s, and from about 10,000 Pa*s to about 22,000 Pa*s according to the Viscosity Test Method. The decrease in viscosity between the neat viscosity and the 25% dilution viscosity can be less than 60%, less than 50%, or less than 45%.

The composition can have a peak viscosity at 50% dilution of from about 500 Pa*s to about 30,000 Pa*s, from about 1000 Pa*s to about 25,000 Pa*s, from about 5000 Pa*s to about 22,000 Pa*s, and from about 10,000 Pa*s to about 20,000 Pa*s, according to the Viscosity Test Method. The decrease in viscosity between the neat viscosity and the 25% dilution viscosity can be less than 75%, less than 65%, less than 60%, or less than 55%.

The body lotion composition can have a retention of viscosity at 25% dilution of greater than 46%, greater than 50%, greater than 52%, and greater than 55%. The body lotion composition can have a retention of viscosity at 50% dilution of greater than 40%, greater than 42%, greater than 45%, and greater than 46%.

TABLE 1

| Product | List of Ingredients |
|---|---|
| Olay ® Firming & | Water, Glycerin, Niacinamide (Vitamin B3), Petrolatum, Isopropyl |
| Hydrating Body Lotion | Isostearate, Caprylic/Capric Triglyceride, Panthenol, Tocopheryl |
| with Collagen | Acetate (Vitamin E), Palmitoyl Pentapeptide-4 (Peptide), Tapioca |
| | Starch, Dimethicone, Stearyl Alcohol, Phenoxyethanol, Cetyl |
| | Alcohol, Sodium Acrylates Copolymer, Benzyl Alcohol, Fragrance, |
| | Behenyl Alcohol, Dimethiconol, Peg-100 Stearate, Cetearyl |
| | Glucoside, Cetearyl Alcohol, Disodium Edta, Ethylhexylglycerin, |
| | Stearic Acid, Palmitic Acid, Polymethylsilsesquioxane, Sodium |
| | Hydroxide, Yellow 5, Red 33. |
| Nivea ® Breathable | Water, Glycerin, C12-15 Alkyl Benzoate, Cetearyl Alcohol, |
| Nourishing Body Lotion | Butyrospermum Parkii (Shea) Butter, Glycine Soja (Soybean) Oil, |
| | Sodium Ascorbyl Phosphate, Tocopherol, PPG-15 Stearyl Ether, |
| | Acrylates/C10-30 Alkyl Acrylate Crosspolymer, Dimethicone, |
| | Phenoxyethanol, Fragrance, Ethylhexylglycerin, Sodium |
| | Hydroxide, Benzoic Acid, BHT. |
| Jergens ® Ultra Healing | Water, Glycerin, Cetearyl Alcohol, Petrolatum, Stearic Acid, C12- |
| Hand and Body Lotion | 15 Alkyl Benzoate, Aluminum Starch Octenylsuccinate, |
| | Dimethicone, Laureth-3, Ceteareth-20, Allantoin, Arginine, Sodium |
| | Hydroxide, Carbomer, Fragrance, Panthenol, Lecithin, Pentylene |
| | Glycol, Alcohol, Ascorbyl Palmitate, Tocopherol, Methylparaben, |
| | Phenoxyethanol, Ethylparaben. |

TABLE 2

| | | | Olay ® | | |
|---|---|---|---|---|---|
| | | | Firming & | Nivea ® | Jergens ® |
| | | | Hydrating | Breathable | Ultra Healing |
| | | | Body Lotion | Nourishing | Hand and |
| | | Example 4 | with Collagen | Body Lotion | Body Lotion |
| Neat | Peak | 36052 | 19235.9 | 6072.3 | 1360.9 |
| | Viscosity | | | | |
| | (Pa*s) | | | | |
| 25% Dilution | Peak | 20379.9 | 2466.88 | 307.6 | 623.65 |
| | Viscosity | | | | |
| | (Pa*s) | | | | |
| | % Change | −43.47 | −87.18 | −94.93 | −54.17 |
| | from Neat | | | | |
| 50% Dilution | Peak | 17075 | 1025.21 | 103.56 | 527.85 |
| | Viscosity | | | | |
| | (Pa*s) | | | | |
| | % Change | −52.64 | −94.67 | −98.29 | −61.21 |
| | from Neat | | | | |
| Retention of | Peak | 565.29 | 12.824 | 5.066 | 45.826 |
| Viscosity @ | Viscosity | | | | |
| 25% Dilution | (Pa*s) | | | | |
| Retention of | Peak | 473.62 | 5.33 | 1.705 | 38.787 |
| Viscosity @ | Viscosity | | | | |
| 50% Dilution | (Pa*s) | | | | |

Table 4 compares the peak viscosity, yield stress, viscosity at 0.1 s⁻¹ and 1 s⁻¹ for Examples 1 and 4 (Table 7, hereafter) and the following commercially available product examples: CeraVe® Moisturizing Face & Body Cream for Normal to Dry Skin, Naturium® Bio-Lipid Restoring Body Lotion, and Aveeno® Daily Moisturizing Cream. The list of ingredients for the commercial products is in Table 3, below. Example 1 had a gel network, but not a lamellar gel network, and the viscosity is driven primarily by the polymer. In Table 4, each example was tested as a neat example and with the addition of 1.5 mL of 4.11 M NaCl to 10 g of the example thoroughly mixed into the example by hand with a spatula. The peak viscosity, yield stress, and the viscosity at 0.1 s⁻¹ and 1 s⁻¹ was measured according to the Viscosity Test Method, described hereafter.

The results in Table 4 indicate that after adding 4.11 M NaCl solution, only Example 4 remains stable, showing no signs of separation or syneresis when viewed under a microscope at 700X. Additionally, Example 4 exhibits a significantly higher peak viscosity and yield stress, which are closer to those of the neat composition, as compared to the other examples tested. This suggests that Example 4 and similar formulations will maintain a pleasant feeling even when exposed to additional salt, whether from perspiration during use and/or from high levels of salt-based active ingredients added to the composition.

TABLE 3

| Product | List of Ingredients |
|---|---|
| CeraVe ® Moisturizing | Aqua/Water, Glycerin, Cetearyl Alcohol, Caprylic/Capric |
| Face & Body Cream for | Triglyceride, Cetyl Alcohol, Ceteareth-20, Petrolatum, Potassium |
| Normal to Dry Skin | Phosphate, Ceramide Np, Ceramide Ap, Ceramide Eop, Carbomer, |
| | Dimethicone, Behentrimonium Methosulfate, Sodium Lauroyl |
| | Lactylate, Sodium Hyaluronate, Cholesterol, Phenoxyethanol, |
| | Disodium Edta, Dipotassium Phosphate, Tocopherol, |
| | Phytosphingosine, Xanthan Gum, Ethylhexylglycerin |
| CeraVe ® Daily | Aqua/Water, Glycerin, Caprylic/Capric Triglyceride, Cetearyl |
| Moisturizing Face and | Alcohol, Cetyl Alcohol, Potassium Phosphate, Ceramide Np, |
| Body Lotion for Normal | Ceramide Ap, Ceramide Eop, Carbomer, Dimethicone, Ceteareth- |
| to Dry Skin | 20, Behentrimonium Methosulfate, Methylparaben, Sodium |
| | Lauroyl Lactylate, Cholesterol, Disodium EDTA, Dipotassium |
| | Phosphate, Propylparaben, Hydrolyzed Hyaluronic Acid, |
| | Phytosphingosine, Xanthan Gum, Polysorbate 20, Polyglyceryl-3 |
| | Diisostearate |
| Aveeno ® Daily | Water, Glycerin, Isopropyl Myristate, Cetyl Alcohol, Carbomer, |
| Moisturizing Cream | Butyrospermum Parkii (Shea) Butter, Avena Sativa (Oat) Kernel |
| | Flour, Potassium Cetyl Phosphate, Caprylyl Glycol, Sodium |
| | Hydroxide, P-Anisic Acid, Ethylhexylglycerin |
| Naturium ® Bio-Lipid | Aqua (Water), Carthamus Tinctorius Seed Oil, Cetyl Alcohol, |
| Restoring Body Lotion | Glycerin, Caprylic/Capric Triglyceride, Butyrospermum Parkii |
| | Butter, Dimethicone, C10-18 Triglycerides, Moringa Oleifera Seed |
| | Oil, Niacinamide, Rosa Canina Fruit Oil, Panthenol, Sodium Pca, |
| | Sodium Stearoyl Glutamate, Squalane, Vaccinium Myrtillus Fruit |
| | Extract, Saccharum Officinarum Extract, Riboflavin, Tocopherol, |
| | Citrus Aurantium Dulcis Fruit Extract, Citrus Limon Fruit Extract, |
| | Sodium Glycolate, Camellia Sinensis Leaf Extract, Spilanthes |
| | Acmella Flower Extract, Sodium Formate, Acer Saccharum Extract, |
| | Sodium Hyaluronate, Cetearyl Olivate, Sorbitan Olivate, |
| | Phenoxyethanol, Carbomer, Tocopheryl Acetate, |
| | Ethylhexylglycerin, Sodium Hydroxide, Tetrasodium Glutamate |
| | Diacetate, Citric Acid, Potassium Sorbate, Sodium Benzoate |

TABLE 4

| | | | CeraVe ® | | | |
|---|---|---|---|---|---|---|
| | | CeraVe ® | Daily | | | |
| | | Moisturizing | Moisturizing | | | |
| | | Face & Body | Face and | Aveeno ® | Naturium ® | |
| | | Cream for | Body Lotion | Daily | Bio-Lipid | |
| | | Normal to | for Normal to | Moisturizing | Restoring | |
| | Example 4 | Dry Skin | Dry Skin | Cream | Body Lotion | Example 1 |
| Emulsifier | Cetyl | Cetearth 20 | Cetearth 21 | Cetyl | Cetearyl | PEG 100 |
| | Phosphate | | | Phosphate | Olicate | stearate |
| | PEG 100 | | | | Sorbitan | Cetearyl |
| | stearate | | | | Olivate | Glucoside |
| Structure | Lamellar Gel | Lamellar Gel | Lamellar Gel | Lamellar Gel | Lamellar Gel | Polymer and |
| Type | Network | Network | Network | Network | Network | Gel Network |

Neat Examples

| | | | | | | |
|---|---|---|---|---|---|---|
| Image | FIG. 2A | FIG. 2B | FIG. 2C | FIG. 2D | FIG. 2E | FIG. 2F |
| Peak | 27244.1 | 13413.2 | 12477.6 | 29802.8 | 12191.8 | 16404.9 |
| Viscosity | | | | | | |
| (Pa*s) | | | | | | |
| Yield | 67.9875 | 27.2978 | 19.6634 | 117.954 | 30.3815 | 40.8107 |
| Stress (Pa) | | | | | | |
| Viscosity | 1032.07 | 446.504 | 187.191 | 2470.2 | 773.713 | 850.878 |
| at 0.1 s⁻¹ | | | | | | |
| (Pa*s) | | | | | | |
| Viscosity | 64.9684 | 59.4302 | 15.753 | 219.529 | 73.8233 | 82.9682 |
| at 1 s⁻¹ | | | | | | |
| (Pa*s) | | | | | | |

After Addition of 1.5 mL of 4.11M NaCl Solution to 10 g of Example

| | | | | | | |
|---|---|---|---|---|---|---|
| Image | FIG. 2G | FIG. 2H | FIG. 2I | FIG. 2J | FIG. 2K | FIG. 2L |
| Peak | 22115.6 | 7209.94 | 4537.28 | 685.814 | 4805.32 | 1656.7 |
| Viscosity | | | | | | |
| (Pa*s) | | | | | | |
| % Change | −18.82 | −46.25 | −63.64 | −97.70 | −60.59 | −89.90 |
| in Peak | | | | | | |
| Viscosity | | | | | | |
| from Neat | | | | | | |
| Yield | 55.1941 | 21.1688 | 11.3531 | 1.72142 | 12.0463 | 4.15436 |
| Stress (Pa) | | | | | | |
| % Change | −18.82 | −22.45 | −42.26 | −98.54 | −60.35 | −89.82 |
| in Yield | | | | | | |
| Stress | | | | | | |
| from Neat | | | | | | |
| Viscosity | 738.548 | 301.406 | 153.048 | 40.6099 | 245.564 | 198.031 |
| at 0.1 s⁻¹ | | | | | | |
| (Pa*s) | | | | | | |
| Viscosity | 65.8562 | 32.7231 | 33.0523 | 2.14266 | 15.4783 | 6.16842 |
| at 1 s⁻¹ | | | | | | |
| (Pa*s) | | | | | | |
| Stable? | Yes | No | No | No | No | No |

The composition can have a peak viscosity after the addition of 1.5 mL of 4.11 M NaCl to 10 g of the example of from about 1000 Pa*s to about 35,000 Pa*s, from about 500 Pa*s to about 30,000 Pa*s, from about 10,000 Pa*s to about 25,000 Pa*s, according to the Viscosity Test Method. The composition can have a % change in peak viscosity from the neat composition to the composition with the 1.5 mL of 4.11 M NaCl to 10 g of the example of about −45% to about 0%, from about −35% to about 0%, from about −30% to about 0%, from about −25% to about 0%, from about −20% to about −5%, from about −19% to about −10% according to the Viscosity Test Method.

The neat composition can have a yield stress of from about 40 Pa to about 110 Pa, from about 50 Pa to about 100 Pa, from about 55 Pa to about 85 Pa, and from about 65 Pa to about 75 Pa, according to the Viscosity Test Method. The composition can have a yield stress after addition of 1.5 mL of 4.11 M NaCl to 10 g of the example of from about 30 Pa to about 100 Pa, from about 40 Pa to about 75 Pa, from about 45 Pa to about 65 Pa, and from about 50 Pa to about 60 Pa according to the Viscosity Test Method. The composition can have a % change in yield stress from the neat composition to the composition with the 1.5 mL of 4.11 M NaCl to 10 g of the example of about −40% to about 0%, from about −35% to about 0%, from about −30% to about 0%, from about −25% to about 0%, from about −22% to about 0%, from about −21% to about −5%, from about −20% to about −7%, and from about −10% to about −19%, according to the Viscosity Test Method.

It was also found that when applied to the skin, the bilayers of the gel network spread, resulting in a film that feels drier and less tacky compared to compositions that lack the gel network, despite having similar levels of emollients and humectants. The improved skin feel can be measured using break time data according to the method described herein.

Compositions that consumers perceive as having a smooth and light weight feeling generally have a change in break time below 1.2 s, a change in break time below 1.1 s, according to the Tack Method, described hereafter. The change in break time can be greater than 0.6 s, greater than 0.7 s, and greater than 0.8 s, according to the Tack Method. If the change in break time is too high, the composition may feel slow to absorb, sticky, and/or like there is a residual film on the skin, which is not ideal for body lotion. However, if the change in break time is too low, the composition will feel too thin, it will be difficult to disperse across the body, and the consumer will not think it is an effective moisturizer. Table 5 shows the change in break time for Examples 2 and 4 (Table 6, hereafter), in addition to commercially available products, which included body lotions, facial moisturizers that are generally formulated so they are a bit heavier feeling than body lotions, and a serum, which generally is not perceived as hydrating. Both Examples 2 and 4 had a change in break time of less than 1.2 s and would likely be considered consumer acceptable.

TABLE 5

| | Change in |
|---|---|
| | Break Time (s) |
| Naturium ® Bio-Lipid Restoring Body Lotion | 1.151 |
| Maëlys ® B-Glossy ® Smoothing Body Serum | 1.115 |
| Olay ® Firming & Hydrating Body Lotion with | 0.532 |
| Collagen | |
| Olay ® Nourishing & Hydrating Body Lotion with | 0.517 |
| Hyaluronic Acid | |
| Aveeno ® Daily Moisturizing Body Lotion | 1.656 |
| Jergens ® Ultra Healing Hand and Body Lotion | 0.579 |
| CeraVe ® Daily Moisturizing Lotion | 0.406 |
| Example 2 | 0.869 |
| Example 4 | 1.059 |
| Olay ® Regenerist ® Micro-Sculpting ® Cream | 0.337 |
| Olay ® Retinol24 ® + Peptide Night Face | 0.946 |
| Moisturizer | |
| Hempz ® Original Herbal Body Moisturizer | 1.156 |
| La Roche-Posay ® Lipikar ® AP + M Triple Repair | 1.313 |
| Body Moisturizing Cream | |
| Vaseline ® Intensive Care ™ Cocoa Radiant ® Lotion | 1.177 |
| EOS ® Shea Better ® 24 HR Moisture Body Lotion | 1.243 |

The skin care skin care composition may exhibit a mean break time of between about 0.01 s and about 1.2 s, between about 0.05 s and about 0.1 s, between 0.08 s and about 0.3 s, and between about 0.10 s and about 0.25 s, as measured by the Tack Method, described hereafter.

Another advantage of the lotion compositions is their enhanced crystalline structures. This structural crystallinity contributes to improved thermodynamic stability, as demonstrated by their notably high phase transition temperatures measured using Differential Scanning Calorimetry (DSC). In practical terms, this means that these lotion compositions can withstand elevated temperatures, such as up to 50° C., which can be encountered during shipping, storage, or consumer use, even in challenging conditions like non-climate-controlled warehouses in scorching desert environments.

The lotion compositions can exhibit phase transition temperatures both from a transition onset and peak measurement exceeding 60° C., which can make the products more robust and resilient. Table 6, below, shows the DSC onset temperature and the DSC peak temperature for Example 4 (Table 7, hereafter), as compared to three commercially available products. The phase transition temperatures were determined according to the DSC Method that was performed as follows: 5 mg samples were placed on the DSC vs. a reference pan. Samples are equilibrated for 3 minutes at 50° C. following which a temperature ramp to 80° C. is performed. A cooling ramp to 20° C. follows with a final temperature ramp to 80° C. A TA® Instruments Discovery Dynamic Scanning calorimeter is used for the measurements. The transition onset and the peak measurement can be determined by looking at the resulting curve.

Table 6, shows the DSC onset transition temperature and the DSC peak temperature is greater than 60° C. for Example 4, which is higher than the phase transition temperatures for the Olay® and the Nivea® body lotions. The Jergens® body lotion also had a DSC onset transition temperature that was less than 60° C. and as discussed above, the Jergens® product did not retain its viscosity when diluted and therefore may be less consumer preferred than Example 4.

TABLE 6

| | | Olay ® | | Jergens ® |
|---|---|---|---|---|
| | | Firming & | Nivea ® | Ultra |
| | | Hydrating | Breathable | Healing |
| | | Body Lotion | Nourishing | Hand and |
| | Ex. 4 | with Collagen | Body Lotion | Body Lotion |
| DSC Onset | 63.68 | 54.13 | 49.54 | 57.86 |
| Transition | | | | |
| Temperature (° C.) | | | | |
| DSC Peak | 67.66 | 59.29 | 52.03 | 65.64 |
| Temperature (° C.) | | | | |

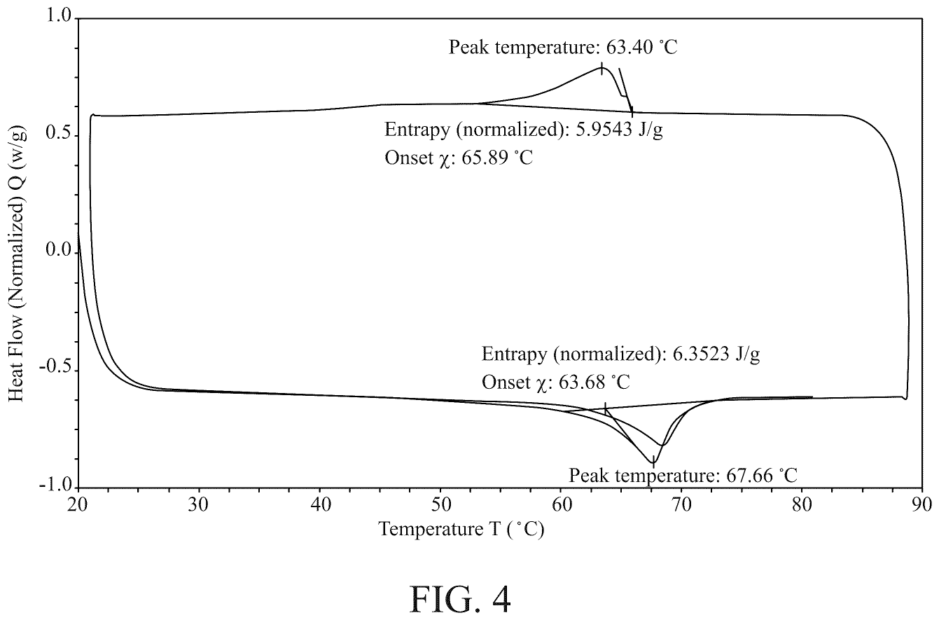

FIG. 4 is a graphic display of the curve created by the DSC method for Example 4. FIG. 3 shows when the sample is exposed to temperatures exceeding the thermal transition temperatures (peak temperature). It also shows that the lamellar gel networks in Example 4 exhibit a significant degree of structural reformation upon cooling, as indicated by the enthalpy normalized values, a secondary peak, shown in FIG. 4. This implies that a significant portion of the original structure is successfully restored, effectively mitigating the detrimental effects of elevated temperatures. In contrast, many traditional systems would experience structure destruction, resulting in product degradation, separation, or syneresis when subjected to temperatures surpassing the phase transition temperature.

The composition can have a DSC Onset Transition Temperature of greater than 58° C., greater than 60° C., greater than 62° C., and greater than 63° C. The composition can have a DSC Peak Temperature of greater than 60° C., greater than 63° C., and greater than 66° C. The DSC Onset Transition Temperature and the DSC Peak Temperature are measured according to the DSC Method, described above. Polymers are used in body lotions to improve the texture and consistency of the product, providing a smooth and spreadable formulation. Additionally, they help to create a protective film on the skin, reducing water loss and improving moisturization, while also enhancing stability and sensory attributes. Cationic polymers can contribute a pleasant feel to skin care moisturizers due to their unique properties and interactions with the skin. However, the lotions described herein include anionic surfactants and anionic systems can interact with the cationic polymers. The composition can contain less than 1%, less than 0.5%, less than 0.25%, less than 0.1%, substantially free of, and free of xanthan gum, modified starches, and/or sodium acrylates copolymer.

It was found that examples with xanthan gum were too gelatinous and stingy and had a tacky texture when applied to skin. Further, it was found that examples that included sodium acrylates copolymer, particularly Caprylic/Capric Triglyceride (and) Sodium Acrylates Copolymer (commercially available as Luvigel® EM from BASF®) felt too greasy, particularly in compositions with relatively high emollient and/or humectant levels. It was also found that compositions that included modified starches, including phosphate modified starch and adipate modified starch, had a custard like texture, which is not consumer preferred for body lotions.

Furthermore, many of the polymers currently used in body lotions are synthetic, which may not align with consumer preferences for natural and sustainable ingredients. Additionally, certain government regulations prioritize the use of natural polymers over synthetic alternatives. However, the use of natural polymers in body lotion formulations often introduces unfavorable attributes to the formula, such as a sticky sensation. This can significantly impact the overall user experience and restrict the applicability of natural polymers in body lotion compositions.

The skin care composition was found to benefit from the inclusion of a biodegradable polymer derived from tara gum and 2-acrylamido-2-methylpropane sulfonic acid (AMPS). This polymer not only provided the composition with a pleasing texture but also imparted a comfortable feel upon application to the skin. Additionally, it exhibited excellent biodegradability, aligning with the increasing demand for environmentally friendly and sustainable cosmetic products. In particular, the polymer can be Caesalpinia spinosa Gum/Ammonium AMPS Crosspolymer (commercially available as Aristoflex® Eco T from Clariant®).

The effectiveness of the Caesalpinia spinosa gum/ammonium AMPS crosspolymer as a rheology modifier in this system was unexpected due to its neutralized cationic nature. Typically, mixing oppositely charged polymers and surfactants is discouraged as it often leads to phase separation. In fact, Clariant®'s Technical Data Sheet for Aristoflex® (issued May 2023) specifically advises against combining this polymer with anionic surfactants.

Contrary to expectations, the Caesalpinia spinosa gum/ammonium AMPS crosspolymer was an effective a rheology modifier in the lotion compositions that included a lamellar gel network. Notably, this polymer stands out due to its ability to impart a light skin feel, which aligns with the preferences of many consumers.

### Composition

The skin care compositions can include an emollient, a humectant, an anionic surfactant, a nonionic surfactant, a fatty atmophile, and/or an aqueous carrier. At least a portion of the anionic surfactant, nonionic surfactant, and the fatty amphiphile form the lamellar gel network that can be dispersed throughout the composition or a portion thereof.

The emollient can be present in a dispersed phase surrounded by a gel network. An emulsifier can promote the formation of the emulsion and can also help maintain product stability.

### Emollient

The composition may include one or more emollients that can be synthetic, natural, naturally derived, and/or biodegradable, all the emollients can be natural, naturally derived, and/or biodegradable. The skin care composition can be formulated without, free of, or substantially free of petroleum-based emollients. The emollient can be a liquid emollient having a melting point below 40° C., below 35° C., or below 30° C. The liquid emollient can be an oil, which can include an ester, alkane, triglyceride, non-volatile silicones, and combinations thereof. In another example, the emollient can be a waxy emollient, such as a fatty alcohol, having a melting point above 40° C., above 50° C. The skin care composition can contain one or more liquid emollients and one more waxy emollients. In some examples, the skin care composition can be substantially free of, formulated without, or free of silicones.

The skin care composition may include from about 0.5% to about 20%, from about 1% to about 15%, from about 3% to about 12%, from about 3.5% to about 11%, from about 5% to about 10% emollient.

### Liquid Emollient

The skin care composition may include from about 0.1% to about 15%, from about 1% to about 9%, from about 1% to about 8%, from about 2% to about 7%, from about 3% to about 6% liquid emollient.

The liquid emollient can be a naturally derived oil that is a plant oil. Examples of the plant oil can include, but are not limited to, palm kernel, coconut, avocado, canola, corn, cottonseed, olive, palm, hi-oleic sunflower, mid-oleic sunflower, sunflower, palm stearin, palm kernel olein, safflower, babassu oils, and combinations thereof. In one embodiment, palm kernel oil may be the selected oil. In another embodiment, coconut oil may be the selected oil. In another embodiment, the plant oil may be a combination of palm kernel oil and coconut oil.

The skin care composition can include one or more of the following liquid emollients:
An alkane or isoalkane, ranging in total carbon from about C8-C60, including but not limited to squalane, squalene, coconut alkanes, coconut/palm kernel alkanes, C9-12 Alkane, C10-13 Alkane, C13-15 Alkane, C15-19 Alkane, C14-22 Alkane, C30-34 Alkane, C9-20 Isoalkane, C10-12 Isoalkane, C12-15 Isoalkane, isohexadecene, C32-54 Isoalkane, or a combination thereof.
An ester formed from an alcohol having from 1-6 carbons and acid having from about 6-22 carbons, including but not limited to isopropyl isostearate, isopropyl palmitate, isoamyl laurate, shea butter ethyl esters, shea butter cetyl esters, shea butter decyl esters, shea butter oleyl esters, jojoba esters, coco-caprylate/caprate, ethyl caprate, ethylhexyl caprate, isoamyl caprylate/caprate, argan oil isostearyl esters, babassu seed oil ethyl esters, Camelina sative seed oil ethyl esters, castor/olive oil esters, coconut oil decyl esters, coconut oil ethyl esters, linseed oil ethyl esters, olive oil decyl esters, olive oil ethylhexyl esters, palm kernel oil triacetin esters, palmitoyl serine/silk amino acid methyl esters, rapeseed oil decyl esters, safflower seed oil decyl esters, sunflower seed oil cetyl esters, sunflower seed oil ethyl esters, trehalose isostearate esters, or a combination thereof.
A triglyceride having 3 carbon chains where the carbon chain length ranges from about C7-C18, including but not limited to triheptanoin, caprylic/capric triglyceride, C10-19 triglycerides, caprylic/capric/lauric triglycerides, hydrogenated C12-C18 triglycerides, triethylhexanoin, or a combination thereof.
A non-volatile silicone fluid including but not limited to dimethicone, amodimethicone, or a combination thereof.

### Waxy Emollient

The skin care composition may include from about 0.1% t about 15%, from about 0.2% to about 10%, from about 0.3% to about 8%, from about 0.5% to about 5%, from about 1% to about 4%, from about 1.5% to about 3.5%, and from about 2% to about 3% waxy emollient.

One or more emollients can be solids at room temperature and have an average carbon chain length ranging from about 12 to about 22, about 12 to about 18, from about 16 to about 22, and 16 to about 18.

The skin care composition can include a waxy emollient, such as a wax, butter, fatty alcohol, or a combination thereof. Waxy emollients can include, but are not limited to, beeswax, jojoba wax, shea butter, palm kernel wax, lanolin wax, shellac wax, rice bran wax, carnauba wax, ozokerite, Cocos nucifera (coconut) butter, goat butter, Acrocomia aculeata seed butter, chiuri butter, carnauba, candelilla, or a combinations thereof. The waxy emollient can include petrolatum and/or derivatives thereof including mineral oil, microcrystalline wax, paraffin oil, or combinations thereof.

### Humectant

The composition can include from about 0.1% to about 20%, from about 2% to about 17%, from about 3% to about 15%, from about 4% to about 13% of one or more humectants.

Suitable humectants can include glycols, diols, polyalkylene glycols and their derivatives. In one aspect, an exemplary class of humectants can include polyhydric alcohols with three or more hydroxyl groups. Suitable polyhydric alcohols can include propylene glycol, sorbitol, hydroxypropyl sorbitol, erythritol, threitol, pentaerythritol, xylitol, glucitol, mannitol, hexane triol (e.g., 1,2,6-hexanetriol), glycerin, ethoxylated glycerine, propoxylated glycerine, or mixtures thereof. In one aspect, the humectant is propylene glycol.

Other suitable humectants can include sodium 2-pyrrolidone-5-carboxylate, guanidine, glycolic acid and glycolate salts (e.g., ammonium and quaternary alkyl ammonium), lactic acid and lactate salts (e.g., ammonium and quaternary alkyl ammonium), hyaluronic acid and derivatives thereof (e.g., salt derivatives such as sodium hyaluronate), urea, sodium pyroglutamate, water-soluble glyceryl poly(meth)acrylate lubricants (such as Hispagel®, available from BASF, Ludwigshafen, Germany), or mixtures thereof.

### Gel Network Phase

The skin care compositions also comprise a gel network phase comprising at least one fatty amphiphile. As used herein, the term “gel network” refers to a lamellar crystalline phase which comprises at least one fatty amphiphile, and at least one secondary component selected from at least one surfactant or an additional fatty amphiphile and water or other suitable solvents. The surfactant can be an anionic and/or nonionic surfactant. The surfactant can include both an anionic and a nonionic surfactant. The lamellar phase can comprise bi-layers made up of a first layer comprising the fatty amphiphile and the surfactant and/or fatty amphiphile and alternating with a second layer comprising the water or other suitable solvent. The term “solid crystalline,” as used herein, refers to the structure of the lamellar phase which forms at a temperature below the chain melt temperature of the layer in the gel network comprising the one or more fatty amphiphile.

The presence of the gel network in the in the final skin care composition in the form of the ELD can be confirmed by means known to one of skill in the art, such as X-ray analysis, optical microscopy (described in FIGS. 1A and 1B and accompanying text), cryo-SEM electron microscopy, and differential scanning calorimetry (described in Table 6 and accompanying text). The Cryo-SEM was performed using a Hitachi FE-SEM SU8230 SN 1632-04 and Gatan Alto Pre-prep as follows: The sample was frozen in liquid ethane in a liquid nitrogen bath to create a planchet. The planchets was transferred to a sample holder. Gatan pre-prep was set at −140° C. and clipping of the planchet was completed and then the clipped planchet was etched for 20 minutes at −95° C. and then coated with gold/palladium for 120 seconds and analyzed on Hitachi set at −140° C.

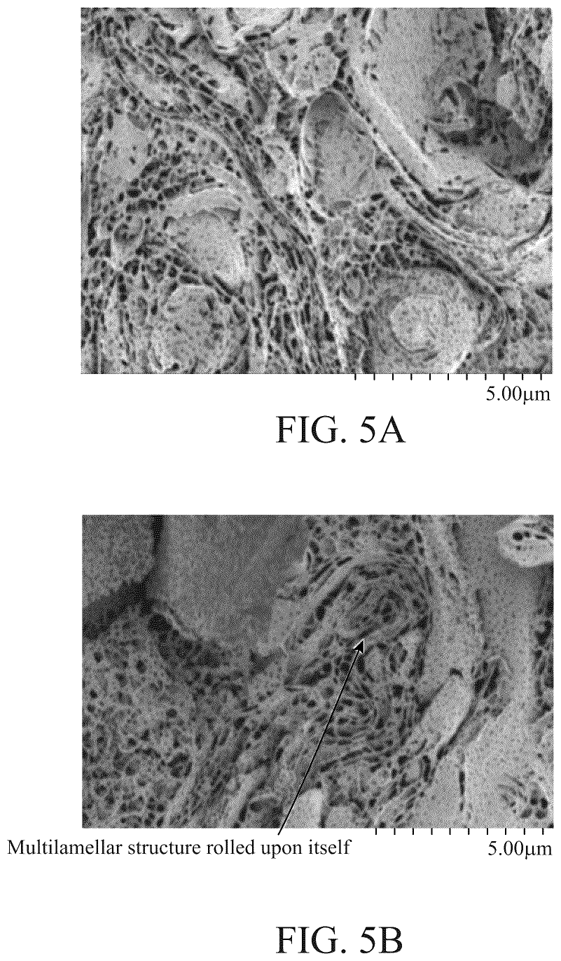

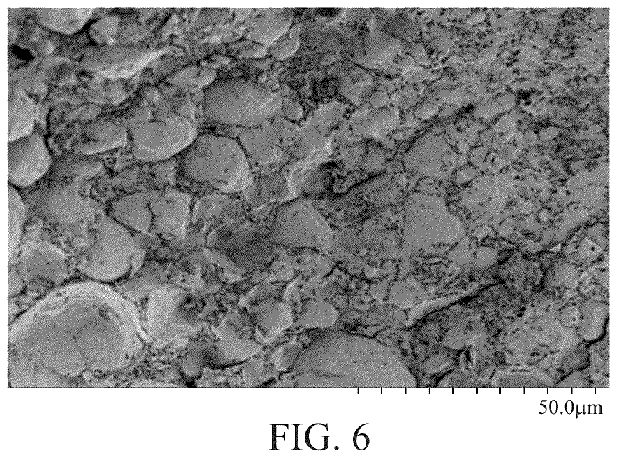

FIGS. 5A and 5B show cryo-SEM (scanning electron microscope) images of a sample of Example 4. The images show multiple lamellar gel network structures that are rolled upon itself, giving the composition it's unique structure. FIG. 6 shows a cryo-SEM image of a sample of Olay® Firming & Hydrating Body Lotion with Collagen. This product has a significantly different structure than Example 4. Notably, no lamellar gel network structure is present and the structure is more micellar.

The aforementioned gel network phase is described as a phase which may be formed by combining one or more fatty amphiphile with one or more surfactants and/or an additional fatty amphiphile with water or a suitable solvent. Suitable solvents other than water include glycerin or other hydrophilic solvents. Therefore, in one embodiment, the gel network phase may be formed by combining one or more fatty amphiphile with one or more surfactants and water, or a suitable solvent. In another embodiment, the gel network phase may be formed by combining one or more fatty amphiphile with one or more additional fatty amphiphiles and water, or a suitable solvent. The term “additional fatty amphiphile” as used herein, means any fatty amphiphile in addition to the first or primary fatty amphiphile.

The weight ratio of the fatty amphiphile to the surfactant in the lamellar gel network component is greater than about 2:1, from greater than about 4:1 to about 100:1, from greater than about 2:1 to about 40:1, and greater than about 2:1 to about 10:1.

The aforementioned ratios can be important for proper and increasingly optimal formation of the gel network phase.

The fatty amphiphile may be combined with both a surfactant and an additional fatty amphiphile to form the aforementioned gel network. In such cases, the aforementioned ratios of fatty amphiphile to the additional fatty amphiphile and of the fatty amphiphile to the surfactant generally apply to gel networks formed by the presence of each of the fatty amphiphile, additional fatty amphiphile and surfactant.

The skin care composition of the present invention comprise a gel network in an amount greater than about 0.1%, from about 1% to about 30%, from about 2% to about 20%, from about 3% to about 10%, and from about 3% to about 8%, by weight of the skin care composition.

### Fatty Amphiphile

The composition can include from about 0.1% to about 15%, from about 0.5% to about 10%, from about 1% to about 8%, from about 1.5% to about 7%, and from about 2% to about 6% fatty amphiphile. The gel network component of the present invention may comprise at least one fatty amphiphile. As used herein, “fatty amphiphile” refers to a compound having a hydrophobic tail group of R₁ as defined below and a hydrophilic head group which does not make the compound water soluble. In general, the material should be soluble at 25° C. at a concentration of 0.1% by weight of the water solvent, preferably at 1%, more preferably at 5%, more preferably at 15%.

The fatty amphiphile may be characterized as a compound having a Hydrophilic-Lipophilic Balance (“HLB”) of 6 or less. The HLB, as used herein, is the standard HLB according to Griffin, J. Soc. Cosm. Chem., vol. 5, 249 (1954).

Suitable fatty amphiphiles, or suitable mixtures of two or more fatty amphiphiles, have a melting point of at least about 27° C. The melting point, as used herein, may be measured by a standard melting point method as described in U.S. Pharmacopeia, USP-NF General Chapter <741> “Melting range or temperature”. The melting point of a mixture of two or more materials is determined by mixing the two or more materials at a temperature above the respective melt points and then allowing the mixture to cool. If the resulting composite is a homogeneous solid below about 27° C., then the mixture has a suitable melting point for use in the present invention. A mixture of two or more fatty amphiphiles, wherein the mixture comprises at least one fatty amphiphile having an individual melting point of less than about 27° C., still is suitable for use in the present invention provided that the composite melting point of the mixture is at least about 27° C.

Suitable fatty amphiphiles can have a hydrophobic tail group of R₁. As used herein, R₁ can be an alkyl, alkenyl (containing up to 3 double bonds), alkyl aromatic, or branched alkyl group of C₁₂-C₇₀ length. The fatty amphiphile can have an average carbon chain length from about 12 to about 22, from about 16 to about 22, from about 16 to about 18, the fatty amphiphile can have an average chain length of from about 12 to about 18. The fatty amphiphile may be saturated or unsaturated. The fatty amphiphiles may be straight or branched. In one example, the waxy emollient may comprise straight chain, saturated fatty alcohol with a terminal hydroxyl.

Non-limiting examples of alkyl, alkenyl, or branched alkyl groups suitable for the fatty amphiphiles of the present invention include lauryl, tridecyl, myristyl, pentadecyl, cetyl, heptadecyl, stearyl, arachidyl, behenyl, undecylenyl, palmitoleyl, oleyl, palmoleyl, linoleyl, linolenyl, arahchidonyl, elaidyl, elaeostearyl, erucyl, isolauryl, isotridecyl, isomyristal, isopentadecyl, petroselinyl, isocetyl, isoheptadecyl, isostearyl, isoarachidyl, isobehnyl, gadoleyl, brassica alcohol, icosyl alcohol, coconut alcohol, hydrogenated jojoba alcohol, jojoba alcohol, and technical-grade mixture thereof.

As used herein, R₁ also may be a branched alkyl group prepared by alkaline condensation of alcohols to give higher molecular weight, branched isoalcohols. These branched isoalcohols are referred to in the art as Guerbet alcohols.

R₁ may be alkyl, alkenyl or branched carbon chains of vegetable origin, such as wheat germ, sunflower, grape seed, sesame, maize, apricot, castor, avocado, olive, soybean, sweet almond, palm, rapeseed, cotton seed, hazelnut, macadamia, karite, jojoba, alfalfa, poppy, pumpkinseed, sesame, cucumber, blackcurrant, evening primrose, millet, barley, quinoa, rye, safflower, candlenut, passion flower or musk rose oil, and karite butter.

Suitable additional fatty amphiphiles also have a hydrophilic head group which does not make the compound water soluble, such as in compounds having an HLB of 6 or less. Non-limiting examples of classes of compounds having such a hydrophilic head group include fatty alcohols, alkoxylated fatty alcohols, fatty phenols, alkoxylated fatty phenols, fatty amides, alkyoxylated fatty amides, fatty amines, fatty alkylamidoalkylamines, fatty alkyoxyalted amines, fatty carbamates, fatty amine oxides, fatty diesters, fatty sorbitan esters, fatty sugar esters, methyl glucoside esters, fatty glycol esters, mono, di & tri glycerides, polyglycerine fatty esters, alkyl glyceryl ethers, propylene glycol fatty acid esters, cholesterol, ceramides, fatty silicone waxes, fatty glucose amides, and phospholipids.

The skin care compositions can be formulated without, substantially free of, or free of cetearyl alcohol. The skin care compositions can be formulated without, substantially free of, or free of palmitic acid and stearic acid.

The fatty amphiphile can include a fatty acid. Suitable fatty acids may be generally defined as fatty acids or alkoxylated fatty acid compounds. More specifically, the fatty acids or alkoxylated fatty acid compounds should generally conform to the following formula:

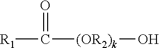

wherein R₁ is as described above; R₂ is a C₁-C₅ carbon chain which can be branched or hydroxy substituted; and k is a number ranging from about 0 to about 5.

Non-limiting examples of suitable fatty acids and alkoxylated fatty acids include behenic acid, stearic acid, C10-40 hydroxyalkyl acid, C32-36 isoalkyl acid coconut acid, erucic acid, hydroxystearic acid, lauric acid, linoleic acid, myristic acid, oleic acid, palmitic acid, PEG-8 behenate, PEG-5 cocoate, PEG-10 cocoate, PEG-2 laurate, PEG-4 laurate PEG-6 laurate, PEG-8 laurate, PEG-9 laurate, PEG-10 laurate, PEG-7 oleate, PEG-2 stearate, PEG-3 stearate, PEG-4 stearate, PEG-5 stearate, PEG-6 stearate, PEG-7 stearate, PEG-8 stearate, PEG-9 stearate, PEG-10 stearate, polyglyceryl-2-PEG-4 stearate, PPG-2 isostearate, and PPG-9 laurate.

### Emulsifier

The skin care composition can contain an emulsifier. The preferred emulsifier can promote the formation of the emulsion, stabilize the composition, and can be chemically and physically compatible with the other components of the composition. The emulsifier can be a surfactant chosen from anionic, cationic, non-ionic, amphiphilic surfactants, or mixtures thereof.

The skin care composition can contain from about 0.10% to about 4%, from about 0.2% to about 3%, from about 0.3% to about 1.5%, from about 0.4% to about 1.25%, from about 0.5% to about 1%, and from about 0.6% to about 0.8% of an emulsifier. The skin care composition can contain less than 2%, less than 1.75%, less than 1.5%, less than 1.25%, less than 1%, and less than 0.9% of an emulsifier.

The emulsifier can be a non-silicone containing emulsifier. Non-limiting examples of non-silicone containing emulsifiers can include polyethylene glycol 20 sorbitan monolaurate (Polysorbate 20), polyethylene glycol 5 soya sterol, Steareth-100, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, cetearyl glucoside, polyglyceryl 10 stearate, and mixtures thereof.

The skin care composition can include a silicone-containing emulsifier. A wide variety of silicone containing emulsifiers can be used. These silicone emulsifiers are typically organically modified organopolysiloxanes which are also known to those skilled in the art as silicone surfactants. Useful silicone emulsifiers include dimethicone copolyols. These materials are polydimethylsiloxanes which have been modified to contain polyether side chains such as polyethylene oxide chains, polypropylene oxide chains, mixtures of these chains, and polyether chains containing groups derived from both ethylene oxide and propylene oxide. Other examples include alkyl-modified dimethicone copolyols, that is, compounds containing pendant C₂-C₃₀ side chains.

Dimethicone copolyols can also be used as an emulsifiers herein including polydimethylsiloxane polyether copolymers with pendant polyethylene oxide sidechains, polydimethylsiloxane polyether copolymers with pendant polypropylene oxide sidechains, polydimethylsiloxane polyether copolymers with pendant mixed polyethylene oxide and polypropylene oxide sidechains, polydimethylsiloxane polyether copolymers with pendant mixed poly(ethylene)(propylene)oxide sidechains, polydimethylsiloxane polyether copolymers with pendant organobetaine sidechains, polydimethylsiloxane polyether copolymers with pendant carboxylate sidechains, polydimethylsiloxane polyether copolymers with pendant quaternary ammonium sidechains; and also further modifications of the preceding copolymers containing pendant C2-C30 straight, branched, or cyclic alkyl moieties. Examples of commercially available dimethicone copolyols useful herein sold by Dow Corning Corporation are Dow Corning® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples of dimethicone copolyols also include lauryl dimethicone copolyol, dimethicone copolyol acetate, dimethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate. See International Cosmetic Ingredient Dictionary, Fifth Edition, 1993, which is incorporated by reference herein in its entirety.

In another example, the emulsifier can include cetearyl olivate and sorbitan olivate (commercially available as Olivem® 1000 from HallStar®) and/or polyglyceryl-3 cetearyl ether olivate (commercially available as Galolive® Bioten from Acme-Hardesty®). It is believed that other emulsifiers that include a mixture of waxy esters made from fatty alcohols and fatty acids derived from natural sources (e.g., olive, palm, corn, palm, rapeseed, soybean, sunflower, coconut, tallow, canola, castor) with a carbon chain length ranging from C12-C22 could be used in stable skin care compositions.

Additional examples of emulsifiers that can be used are found in U.S. Pat. No. 6,174,533, incorporated herein by reference.

### Anionic Surfactant

The composition can include from about 0.05% to about 1.5%, from about 0.1% to about 1%, from about 0.25% to about 0.75%, from about 0.33% to about 0.66%, from about 0.37% to about 0.6%, from about 0.4% to about 0.5%, and from about 0.42% to about 0.48% of an anionic surfactant. The anionic surfactant can comprise an alkyl phosphate monoester and/or diester from the range of C8 to C30 and their salts. These are also described as mono- and/or dicetyl esters of phosphoric acid or salts their salts. One such example would be potassium cetyl phosphate (CAS-No.: 19035-79-1) which has the empirical formula C16H3404PK and the structural formula as shown.

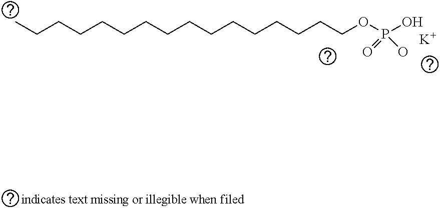 [SMILES: CCCCCCCCCCCCCCCCOP(=O)(O)O ; [K+]]

### Nonionic Surfactant

The composition can include from about 0.1% to about 1%, from about 0.2% to about 0.5%, and from about 0.3% to about 0.5% nonionic surfactant. Nonionic surfactants can include including linear alcohol ethoxylate, secondary alcohol ethoxylates, ethoxylate esters or alkyl polyglycerides. One or more polyethyleneoxide chains wherein each polyethyleneoxide chain contains on average at least about 5 ethylene oxide units and a maximum of 200 polyethylene Oxide units. This include polyoxyethylene alkyl ethers, polyethyleneglycol fatty acid esters, polyoxyethylene castor oil, polyoxyethylene hydrogenated castor oil, polyoxyethylene fatty amides and their monoethanolamine and diethanolamine derivatives, and polyethoxylated fatty amines. It further includes esters of polyethylene glycol and C8-C30 fatty Acids for example PEG-100 Stearate. Among preferred nonionic surfactants comprising one or more polyethyleneoxide chain include polyoxyethylene alkyl ethers having at least about 5, preferably from about 10 to 200, ethylene oxide units. Examples of such nonionic surfactants are steareth-100 and PEG 100 Stearate. The non-ionic surfactants can include alkyl polyglycerides and/or esters of alkyl polyglycerides, such as polyglyceryl 10 stearate.

### Polymer

The composition can optionally include a polymer. If present, the composition can include from about 0.05% to about 2%, from about 0.1% to about 1.5%, from about 0.1% to about 1.25%, from about 0.1% to about 1%, and from about 0.25% to about 0.6% polymer. The composition can include a polymer chosen from Caesalpinia spinosa gum/ammonium AMPS Crosspolymer (commercially available as Aristoflex® Eco T from Clariant®), starch acetate/adipate (and) citric acid (commercially available as Carbopol® Fusion S-20 from Lubrizol®), water (and) alpha-glucan hydroxypropyltrimonium chloride (and) propylene glycol (commercially available as AURIST™ AGC), Cyamopsis tetragonoloba (guar) gum (commercially available as AURIST™ GHI), hydroxyethyl acrylate/sodium acryloyldimethyl taurate copolymer (commercially available as SEPINOV™ EMT 10), sodium polyacryloyldimethyl taurate (commercially available as Aristoflex® Silk from Clariant®), carboxymethyl chitosan (commercially available as CHITOGLYCAN PF), Sphingomonas ferment extract (commercially available as Kelco-Care™ diutan gum from Lubrizol®), acrylamide/sodium acryloyldimethyltaurate copolymer (and) C15-19 Alkane (and) polyglyceryl-10 laurate (and) polyglycerin-10 (commercially available as SEPILIFE™ G305, or mixtures thereof.

### Dermatologically Acceptable Carrier

The continuous phase can include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. The carrier can be present at a level of from about 50% to about 95%, about 52% to about 90%, about 55% to about 85%, or, from about 60% to about 80%, by weight of the composition.

The carrier can be in a wide variety of forms. In some instances, the solubility or dispersibility of the components (e.g., extracts, sunscreen active, additional components) may dictate the form and character of the carrier. The product form can include dispersions, emulsions, and solid forms (e.g., sticks, flowable solids, amorphous materials). The skin care composition can be aqueous or anhydrous. The oil phase of the emulsion may include silicone oils, esters, ethers, triglycerides and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). The composition can contain from about 1% to about 20%, from about 3% to about 18%, from about 5% to about 17%, and from about 6% to about 15% humectant.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more non-volatile oils (e.g., botanical oils and/or silicone oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, glycerol, butylene glycol, 1,2,4-butanetriol, sorbitol esters, 1,2,6-hexanetriol, ethanol, isopropanol, sorbitol esters, butanediol, ether propanol, and combinations thereof.

### Vitamin B₃ Compound

The compositions herein can include a safe and effective amount of a vitamin B₃ compound. The Vitamin B₃ compound can be in the continuous phase. In some instances, the present compositions may contain 0.01% to 10%, by weight, of the vitamin B₃ compound, based on the weight or volume of the composition (e.g., 0.1% to 10%, 0.5% to 5%, or even 1% to 3%). As used herein, “vitamin B₃ compound” means a compound having the formula:

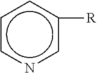

Where: R is CONH₂ (i.e., niacinamide), COOH (i.e., nicotinic acid) or CH₂OH (i.e., nicotinyl alcohol); derivatives thereof; and salts of any of the foregoing.

Exemplary derivatives of vitamin B3 compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide.

### Other Optional Ingredients

The present composition may optionally include one or more additional ingredients commonly used in cosmetic compositions (e.g., dyes, pigments, mica, insoluble particles, skin care actives, anti-inflammatory agents, sunscreen agents, emulsifiers, buffers, rheology modifiers, chelants, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. In some examples, the composition can include a fragrance, in particular a natural fragrance, or a colorant, in particular a natural colorant. In some examples, the skin care composition can include a silicone elastomer including but not limited to silicone elastomers supplied solvated in non-volatile linear silicone fluids, silicone elastomers supplied solvated in alkanes, esters or triglycerides. The chelant can be selected from ethylenediaminetetraacetic acid (EDTA), trisodium ethylenediamine disuccinate (EDDS), trisodium dicarboxymethyl alaninate, tetrasodium glutamate diacetate, phytic acid, or a combination thereof. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

In some examples, the composition can be formulated without, substantially free of, or free of dyes. In some examples, the composition can be formulated without, substantially free of, or free of fragrance materials.

Since the composition can have a high salt tolerance, it can be stable with a relatively high level (e.g., from about 5% to about 10%, about 6% to about 9%, about 7% to about 8%) of skin care active ingredients that contain salt. Examples of skin care actives that contain salt can include sodium hyaluronate, sodium salicylate, zinc sulfate, sodium ascorbyl phosphate, sodium citrate, niacinamide, or combinations thereof.

### Method of Use

The skin care lotion composition may be applied to the entire body or portions of the body including the arms, legs, back, face, neck, hands, feet, and/or a portion or combination thereof at least once a day, twice a day, or on a more frequent daily basis, as needed, during a treatment period. The treatment period herein is ideally of sufficient time for the skin care actives to improve the appearance of the skin. The treatment period may last for at least a week (e.g., about 1 week, about 2 weeks, about 4 weeks, about 8 weeks, or even about 12 weeks). In some instances, the treatment period will extend over multiple months (i.e., about 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The skin care composition can be applied in one or more of the following ways:
After showering or bathing: This means that they can directly apply it to their wet or damp skin without drying themselves off with a towel. If desired, they can towel dry if they still feel wet after applying the composition.
After towel drying: The user can apply the skin care composition soon after taking a shower or bath. The user can dry themselves with a towel until their skin feels completely dry or the skin can still be slightly damp and then they can apply the body lotion to the desired areas.

The skin care composition can be fast-absorbing and can penetrate skin up to 10 layers deep. The skin care composition can be used on all types of skin. The skin care composition can hydrate skin and/or increase cell turnover. The formula can be moisturizing and can feel lightweight. The composition can hydrate skin with plumping moisture that can tightens and can diminishes fine lines. The composition can leave skin feeling softer and smoother. The skin care composition can be used in combination with body wash that can boost the effects.

### Definitions

As used herein, “biodegradable” refers to materials that meet the pass levels for readily and ultimate biodegradability according to the OECD Guideline for Testing of Chemicals, Method 301 B: CO2 Evolution (Modified Sturm Test) (adopted Jul. 17, 1992).

As used herein, “body lotion” or “lotion” includes skin care products that are topically applied to regulate or improve a skin care condition, particularly by providing hydration and/or moisturization. As used herein, the term “lotion” or “body lotion” can refer to moisturizers, creams, body butter, body milk, or the like.

As used herein, “formulated without” means that the ingredient is not intentionally added. However, “formulated without” does not guarantee “100% free from” since trace contaminants are possible.

“Improve the appearance of” means providing a measurable, desirable change or benefit in skin appearance, which may be quantified, for example, by a decrease in redness, inflammation, and/or plaque scales.

As used herein, “molecular weight” refers to weight average molecular weight unless otherwise stated. Molecular weights are measured using the industry standard method of gel permeation chromatography (“GPC”).

As used herein, “natural” refers to cosmetic ingredients obtained only from plants, animals, micro-organisms, or minerals according to International Organization for Standardization 16128-1, Part 1, ¶2.1 (Feb. 15, 2016). Ingredients obtained from fossil fuels are excluded from the definition. A non-limiting list of “natural” ingredient sources can include olive, palm, corn, palm, rapeseed, soybean, sunflower, coconut, tallow, canola, castor, and combinations hereof.

As used herein, “naturally derived” refers to cosmetic ingredients of greater than 50% natural origin by molecular weight, by renewable carbon content or by any other relevant methods, obtained through defined chemical and/or biological processes with the intention of chemical modification according to ISO 16128-1, Part 1, ¶3.1 (Feb. 15, 2016). ISO 16128-1, Appendix B includes a non-limiting list of chemical and biological processes for derived natural ingredients, which is incorporated by reference.

As used herein, “skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin; preventing damage to skin via antioxidant approaches, including UV A and UV B induced damage, preventing formation of comedomes, balancing the skin microbiome or preventing acne.

As used herein, “skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

As used herein, “skin care composition” means a composition that includes a skin care active and regulates and/or improves skin condition.

As used herein, “substantially free” means less than 0.5%, less than 0.25%, less than 0.1%, less than 0.05%, and/or less than 0.01%.

As used herein, “treatment period,” means the length of time and/or frequency that a material or composition is applied to a target skin surface.

Other than in the operating examples, or where otherwise indicated, all numbers expressing quantities of ingredients and/or reaction conditions are to be understood as being modified in all instances by the term “about” which can encompass ±10%, 8%, ±6%, ±5%, ±4%, ±3%, ±2%, ±1%, or ±0.5%.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

It is to be understood that, as used herein the terms “the,” “a,” or “an,” mean “at least one,” and should not be limited to “only one” unless explicitly indicated to the contrary.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Test Methods

### Tack Method

This method provides a suitable means for determining the tackiness of the compositions described herein. The method uses a texture analyzer to contact a probe with a film formed from the composition. The texture analyzer then measures the force needed to separate the probe from the composition film. Tack Force can be determined by this method. The Tack Method is configured to run for a period of 100 minutes. It is believed, without being limited by theory, that the tackiness properties exhibited by a composition over the course of the Tack Test approximate the tackiness properties exhibited by the composition during the first few minutes (e.g., less than 10 minutes, between 30 seconds and 5 minutes, or between 1 minute and 3 minutes) of use of the composition by a user.

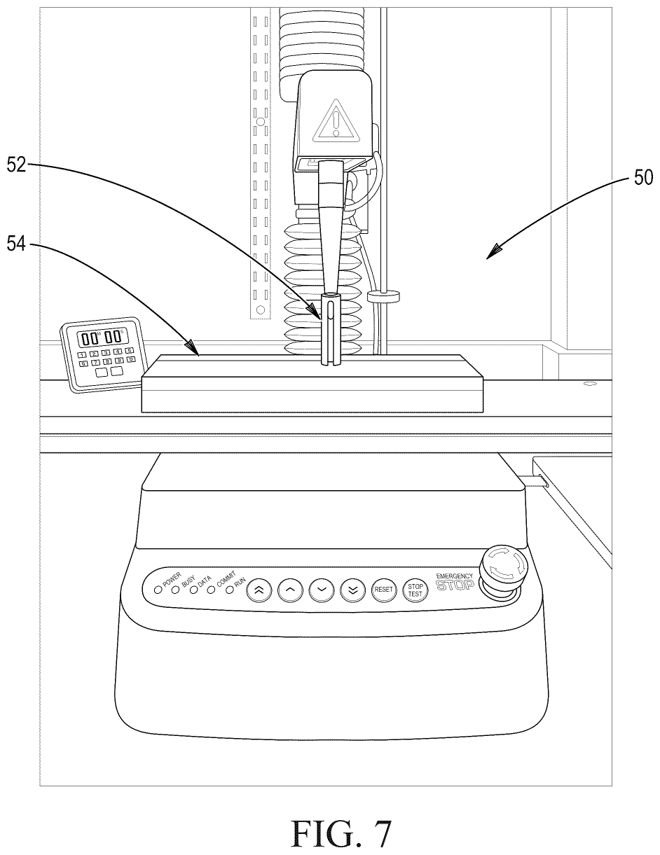

FIG. 7 illustrates and exemplary test setup for the Tack Test. A TA.XT2i brand texture analyzer (available from Texture Technologies Corporation, MA) 50 or equivalent is used to measure the tack of a film formed from a composition of interest. The texture analyzer is equipped with a tack probe 52 in the form of a 12.75 mm diameter acrylic cylinder with a flat surface. During testing, the flat surface of the probe 52 is contacted with the surface of a film of the composition. Thus, the flat surface of the probe and the surface of the film must be parallel to one another during testing to ensure there is sufficient contact by the film across the effective test surface of the probe 52. The film is made by filling or overfilling (followed by draw down) a rectangular channel 54 (e.g., 25 cm long×30 mm wide a 0.25 mm deep) with the composition to be tested.

The test is conducted using an adhesive test protocol with a pretest speed of 0.10 mm/second, a test speed of 0.10 mm/see and a post-test speed of 1.0 mm/sec. The applied force is 200 g, the return distance is 4 mm and the contact time is 5.0 sec. The trigger type to designate sample contact is set to automatic and the trigger force is 5.0 g. The test is run and at the following time increments immediately after the film is prepared: <1 minute (i.e., immediately following preparation of the film), 10, 20, 30, 40, 50, 60, 80, and 100 minutes. Each time point is run on a previously undisturbed/untested area of the sample. Each sample is run in triplicate and the averages are recorded.

Break Time is determined by the width of the force curve. The start time of the Break Time calculation is when the sign of the force exerted by the texture analyzer changes to positive from negative at time zero (i.e., the start of the test) and the time at which the force decays back to 0.0+/−0.02 from the peak force as the break time. Mean Break Time is reported as the average of the break times at 60, 80 and 100 minutes. The Change in Break Time is the difference between the initial Break time and the Break time at 100 minutes.

### Viscosity Test Method

First, the samples were prepared. If the sample was diluted, water or NaCl solution was added to the desired dilution by means of gravimetry and mixed in a beaker for 3 minutes.

The viscosities of the examples are measured by a Discovery HR-2 Hybrid rheometer (TA® Instruments) using a stainless steel, cross hatched, 40 mm plate (available from TA® Instruments) at 25° C., gap 1000 μm. A flow sweep was performed from 0.001 to 500 s⁻¹ shear rate in logarithmic sample mode following 3 minutes of equilibration time at 25° C. The peak viscosity is the point where the viscosity is the highest. Viscosity at 0.1 s⁻¹ is the viscosity measured at the shear rate of 0.1 s⁻¹. Viscosity at 1 s⁻¹ is the viscosity measured at the shear rate of 0.1 s⁻¹. The yield stress is the stress measured at peak viscosity, as measured by the rheometer.

### EXAMPLES

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

The Examples in the Tables, below, were prepared by first combining the water phase ingredients (e.g., carrier and humectant) in a container and mixing until uniform and anionic surfactant, if present, are added to the water phase. For Examples 2-15, which have a lamellar gel network, the dispersed phase (all other materials except carrier, humectant, some actives, anionic surfactant, and certain polymers) including the biodegradable polymer are combined in a separate container and mixed until uniform. For Examples 1 and 16, the dispersed phase (all other materials except carrier, humectant, some actives, anionic surfactant, and the polymers) are added in a separate container and mixed until uniform. Each of the phases are heated to ˜75° C. The dispersed phase and optionally additional polymers (except the Luvigel® in Example 1) are then added to the water phase with high shear mixing (e.g., Rotor Stator high shear mixer) to form an emulsion. The emulsion is then cooled and continuously mixed until it reaches a temperature below −40° C. Then, heat sensitive actives, if present Luvigel®, and other volatile ingredients (e.g., retinyl propionate, preservatives, perfumes) may be added, if present, and optionally the composition is mixed again with high shear mixing. Finally, the composition can be poured into a container for storage, use, and/or testing.

TABLE 7

| | | Ex. 1 | Ex. 2 | Ex. 3 | Ex. 4 | Ex. 5 | Ex. 6 |
|---|---|---|---|---|---|---|---|
| | | (wt. %) | (wt. %) | (wt. %) | (wt. %) | (wt. %) | (wt. %) |
| Carrier | Water | Q.S. | Q.S. | Q.S. | Q.S. | Q.S. | Q.S. |
| Humectant | Glycerin | 11.000 | 9.000 | 11.000 | 7.000 | 5.000 | 5.000 |
| Emollients | Glyceryl | 0.000 | 0.750 | 0.750 | 1.250 | 0.750 | 0.500 |
| | Monostearate | | | | | | |
| | Glyceryl | 0.000 | 0.000 | 0.000 | 0.000 | 0.000 | 0.000 |
| | Monooleate | | | | | | |
| | Caprylic/Capric | 0.600 | 2.250 | 0.600 | 1.000 | 2.250 | 1.250 |
| | Triglyceride¹ | | | | | | |
| | Petrolatum | 2.000 | 2.000 | 2.000 | 2.000 | 0.000 | 0.000 |
| | Isopropyl | 1.500 | 1.500 | 1.500 | 1.500 | 1.500 | 1.500 |
| | Isostearate | | | | | | |
| | Coco- | 0.000 | 0.000 | 0.000 | 0.000 | 0.000 | 0.000 |
| | Caprylate/Caprate² | | | | | | |
| | Hydrolyzed Jojoba | 0.000 | 0.000 | 0.000 | 0.000 | 0.000 | 0.000 |
| | Esters (and) Jojoba | | | | | | |
| | Esters (and) Water³ | | | | | | |
| | Butyrospermum | 0.000 | 0.000 | 0.700 | 3.000 | 0.000 | 0.000 |
| | parkii (Shea Butter) | | | | | | |
| | Dimethicone (and) | 0.500 | 1.500 | 1.000 | 1.000 | 0.500 | 0.500 |
| | Dimethiconol | | | | | | |
| Structurants/ | Caprylic/Capric | 2.000 | 0.000 | 0.000 | 0.000 | 0.000 | 0.000 |
| Thickeners | Triglycerides⁴ | | | | | | |
| | xanthan gum | | | | | | |
| | Caesalpinia | 0.000 | 0.150 | 0.325 | 0.300 | 0.150 | 0.150 |
| | Spinosa | | | | | | |
| | Gum/Ammonium | | | | | | |
| | AMPS | | | | | | |
| | Crosspolymer⁴ | | | | | | |
| | Stearyl Alcohol | 0.641 | 0.000 | 0.600 | 1.120 | 0.000 | 0.000 |
| | Cetyl Alcohol | 0.515 | 0.000 | 1.060 | 2.500 | 0.000 | 0.000 |
| | Behenyl Alcohol | 0.420 | 0.200 | 0.600 | 1.120 | 0.200 | 0.200 |
| Powders | Tapioca Starch⁵ | 0.500 | 0.750 | 1.250 | 0.500 | 0.750 | 0.750 |
| pH Adjuster | | pH adjusted to ~5.5-5.7 | | | | | |
| Emulsifiers | Cetearyl Alcohol | 0.000 | 3.500 | 0.000 | 0.000 | 3.500 | 3.000 |
| | (and) Dicetyl | | | | | | |
| | Phosphate (and) | | | | | | |
| | Ceteth-10 | | | | | | |
| | Phosphate⁶ | | | | | | |
| | Cetearyl Glucoside | 0.200 | 0.000 | 0.000 | 0.000 | 0.000 | 0.000 |
| | (and) cetearyl | | | | | | |
| | alcohol | | | | | | |
| | Stearic Acid | 0.100 | 0.000 | 0.150 | 0.200 | 0.000 | 0.000 |
| | Polyglceryl 10 | 0.000 | 0.000 | 0.000 | 0.000 | 0.000 | 0.000 |
| | Stearate | | | | | | |
| | Potassium Cetyl | 0.000 | 0.000 | 0.462 | 0.500 | 0.000 | 0.000 |
| | Phosphate | | | | | | |
| | PEG-100 Stearate | 0.100 | 0.100 | 0.186 | 0.400 | 0.100 | 0.100 |
| Chelator | Disodium EDTA | 0.100 | 0.100 | 0.100 | 0.100 | 0.100 | 0.100 |
| Preservatives | Benzyl Alcohol | 0.400 | 0.400 | 0.400 | 0.400 | 0.400 | 0.400 |
| | Phenoxyethanol | 0.750 | 0.750 | 0.750 | 0.750 | 0.750 | 0.750 |
| | (and) | | | | | | |
| | Ethylhexylglycerin | | | | | | |
| Skin | Niaciniamide | 4.000 | 3.000 | 3.000 | 3.000 | 3.000 | 3.000 |
| Conditioning | Panthenol | 0.250 | 0.250 | 0.250 | 0.250 | 0.250 | 0.250 |
| Actives | | | | | | | |
| Fragrance | Perfume | 0.300 | 0.000 | 0.400 | 0.000 | 0.000 | 0.000 |
| Colorant | Dye | 0.000 | 0.000 | 0.000 | 0.000 | 0.000 | 0.000 |
| Actives | Vitamin E | 0.011 | 0.000 | 0.000 | 0.000 | 0.000 | 0.000 |
| | Hyaluronic Acid | 0.005 | 0.000 | 0.000 | 0.000 | 0.000 | 0.000 |
| | Collagen | 0.000 | 0.000 | 0.000 | 0.000 | 0.000 | 0.000 |
| | Trehalose | 0.000 | 0.000 | 1.000 | 0.000 | 0.000 | 0.000 |
| | Vitamin C | 0.000 | 0.000 | 0.000 | 0.000 | 0.000 | 0.000 |

TABLE 8

| | | Ex. 7 | Ex. 8 | Ex. 9 | Ex. 10 | Ex. 11 | Ex. 12 |
|---|---|---|---|---|---|---|---|
| | | (wt. %) | (wt. %) | (wt. %) | (wt. %) | (wt. %) | (wt. %) |
| Carrier | Water | Q.S. | Q.S. | Q.S. | Q.S. | Q.S. | Q.S. |
| Humectant | Glycerin | 5.000 | 5.000 | 11.000 | 11.000 | 11.000 | 11.000 |
| Emollients | Glyceryl | 0.500 | 0.500 | 0.750 | 0.750 | 0.750 | 0.750 |
| | Monostearate | | | | | | |
| | Glyceryl | 0.000 | 0.000 | 0.000 | 0.000 | 0.000 | 0.000 |
| | Monooleate | | | | | | |
| | Caprylic/Capric | 1.250 | 1.250 | 0.600 | 0.600 | 0.600 | 0.600 |
| | Triglyceride¹ | | | | | | |
| | Petrolatum | 0.000 | 0.750 | 2.000 | 0.000 | 0.000 | 2.000 |
| | Isopropyl Isostearate | 1.500 | 1.500 | 1.500 | 0.000 | 0.000 | 1.500 |
| | Coco- | 0.000 | 0.000 | 0.000 | 1.500 | 1.500 | 0.000 |
| | Caprylate/Caprate² | | | | | | |
| | Hydrolyzed Jojoba | 0.000 | 0.000 | 0.000 | 2.000 | 2.000 | 0.000 |
| | Esters (and) Jojoba | | | | | | |
| | Esters (and) Water³ | | | | | | |
| | Butyrospermum | 0.000 | 0.000 | 0.700 | 0.700 | 0.700 | 0.700 |
| | parkii (Shea Butter) | | | | | | |
| | Dimethicone (and) | 0.500 | 0.500 | 1.000 | 1.000 | 1.000 | 1.000 |
| | Dimethiconol | | | | | | |
| Structurants/ | Caprylic/Capric | 0.000 | 0.000 | 0.000 | 0.000 | 0.000 | 0.000 |
| Thickeners | Triglycerides | | | | | | |
| | xanthan gum | | | | | 0.750 | |
| | Caesalpinia Spinosa | 0.150 | 0.150 | 0.000 | 0.150 | 0.000 | 0.325 |
| | Gum/Ammonium | | | | | | |
| | AMPS | | | | | | |
| | Crosspolymer⁴ | | | | | | |
| | Stearyl Alcohol | 0.000 | 0.000 | 0.600 | 0.600 | 0.600 | 0.600 |
| | Cetyl Alcohol | 0.000 | 0.000 | 1.060 | 1.060 | 1.060 | 1.060 |
| | Behenyl Alcohol | 0.000 | 0.000 | 0.600 | 0.600 | 0.600 | 0.600 |
| Powders | Tapioca Starch⁵ | 0.750 | 0.750 | 1.250 | 1.250 | 1.250 | 1.250 |
| pH Adjuster | | pH adjusted to ~5.5-5.7 | | | | | |
| Emulsifiers | Cetearyl Alcohol | 2.500 | 2.500 | 0.000 | 0.000 | 0.000 | 0.000 |
| | (and) Dicetyl | | | | | | |
| | Phosphate (and) | | | | | | |
| | Ceteth-10 | | | | | | |
| | Phosphate⁶ | | | | | | |
| | Cetearyl Glucoside | 0.000 | 0.000 | 0.000 | 0.000 | 0.000 | 0.000 |
| | (and) cetearyl | | | | | | |
| | alcohol | | | | | | |
| | Stearic Acid | 0.000 | 0.000 | 0.150 | 0.150 | 0.150 | 0.150 |
| | Polyglceryl 10 | 0.000 | 0.000 | 0.000 | 0.000 | 0.000 | 0.200 |
| | Stearate | | | | | | |
| | Potassium Cetyl | 0.000 | 0.000 | 0.462 | 0.462 | 0.462 | 0.462 |
| | Phosphate | | | | | | |
| | PEG-100 Stearate | 0.100 | 0.100 | 0.186 | 0.186 | 0.186 | 0.000 |
| Chelator | Disodium EDTA | 0.100 | 0.100 | 0.100 | 0.100 | 0.100 | 0.100 |
| Preservatives | Benzyl Alcohol | 0.400 | 0.400 | 0.400 | 0.400 | 0.400 | 0.400 |
| | Phenoxyethanol | 0.750 | 0.750 | 0.750 | 0.750 | 0.750 | 0.750 |
| | (and) | | | | | | |
| | Ethylhexylglycerin | | | | | | |
| Skin | Niaciniamide | 3.000 | 3.000 | 3.000 | 3.000 | 3.000 | 3.000 |
| Conditioning | Panthenol | 0.250 | 0.250 | 0.250 | 0.250 | 0.250 | 0.250 |
| Actives | | | | | | | |
| Fragrance | Perfume | 0.000 | 0.000 | 0.400 | 0.400 | 0.400 | 0.400 |
| Colorant | Dye | 0.000 | 0.000 | 0.000 | 0.000 | 0.000 | 0.000 |
| Actives | Vitamin E | 0.000 | 0.000 | 0.000 | 0.000 | 0.000 | 0.000 |
| | Hyaluronic Acid | 0.000 | 0.000 | 0.000 | 0.000 | 0.000 | 0.000 |
| | Collagen | 0.000 | 0.000 | 0.000 | 0.000 | 0.000 | 0.000 |
| | Trehalose | 0.000 | 0.000 | 1.000 | 1.000 | 1.000 | 1.000 |
| | Vitamin C | 0.000 | 0.000 | 0.000 | 0.000 | 0.000 | 0.000 |

TABLE 9

| | | Ex. 13 | Ex. 14 |
|---|---|---|---|
| | | (wt. %) | (wt. %) |
| Carrier | Water | Q.S. | Q.S. |
| Humectant | Glycerin | 11.000 | 7.000 |
| Emollients | Glyceryl Monostearate | 1.500 | 0.000 |
| | Glyceryl Monooleate | | |
| | Caprylic/Capric Triglyceride¹ | 1.000 | 6.000 |
| | Petrolatum | 2.000 | |
| | Isopropyl Isostearate | 1.500 | |
| | Coco-Caprylate/Caprate/ | | 2.000 |
| | C12-14 Alkanes | | |
| | Hydrolyzed Jojoba Esters | | |
| | (and) Jojoba Esters (and) | | |
| | Water³ | | |
| | Butyrospermum parkii (Shea | 3.000 | |
| | Butter) | | |
| | Dimethicone (and) | 1.000 | |
| | Dimethiconol | | |
| Structurants/ | Caprylic/Capric Triglycerides | | |
| Thickeners | xanthan gum | | |
| | Caesalpinia Spinosa | 0.300 | |
| | Gum/Ammonium AMPS | | |
| | Crosspolymer⁴ | | |
| | Stearyl Alcohol | 1.120 | 1.500 |
| | Cetyl Alcohol | 2.500 | 2.000 |
| | Behenyl Alcohol | 1.120 | 1.500 |
| Powders | Zea Mays Corn Starch | | 2.000 |
| pH Adjuster | | pH adjusted to ~5.5-5.7 | |
| Emulsifiers | Cetearyl Alcohol (and) | | |
| | Dicetyl Phosphate (and) | | |
| | Ceteth-10 Phosphate⁶ | | |
| | Cetearyl Glucoside (and) | | |
| | cetearyl alcohol | | |
| | Stearic Acid | 0.200 | |
| | Polyglyceryl-3 Methylglucose | | 2.000 |
| | Distearate | | |
| | Potassium Cetyl Phosphate | 0.500 | |
| | PEG-100 Stearate | 0.400 | |
| Chelator | Disodium EDTA | 0.100 | |
| Preservatives | Sodium Benzoate | | 0.200 |
| | Benzyl Alcohol | 0.400 | 0.900 |
| | Phenoxyethanol (and) | 0.750 | |
| | Ethylhexylglycerin | | |
| Skin | Niaciniamide | 4.000 | 2.000 |
| Conditioning | Panthenol | 0.250 | 0.500 |
| Actives | | | |

Suppliers for Table 7, Table 8, and Table 9:
1. Myritol® 318 from BASF®
2. Cetiol® LC by BASF®
3. Liponate® Jojoba NatFilm from Vantage Personal Care
4. Aristoflex® Eco T from Clariant®
5. Dry-Flo® TS
6. Crodafos™ CES from Croda Inc.

### Clinical Test:

The examples in Table 10 were placed in a clinical trial with 22 female participants aged 18-75, selected from a pre-screened pool of 30 based on the highest baseline transepidermal water loss (TEWL) values. Participants were required to be in good health with dry to normal skin types, while those with sensitive skin were excluded.

TABLE 10

| Leg | Example | Code | Structure Type |
|---|---|---|---|
| 1 | Example 1 (Table 7) | A | Polymer and Gel |
| | | | Network |
| 2 | Example 4 (Table 7) | B | Lamellar Gel Network |
| 3 | Example 13 (Table 9) | C | Lamellar Gel Network |
| 4 | Example 14 (Table 10) | D | Polymer and Gel |
| | | | Network |
| 5 | CeraVe ® Daily Moisturizing Face | E | Lamellar Gel Network |
| | and Body Lotion for Normal to | | |
| | Dry Skin (Table 3) | | |
| 6 | No Treatment | F | n/a |

In the clinical study, The Delfin® VapoMeter® measured transepidermal water loss (TEWL) from the skin's surface.

Before beginning the study, participants underwent a 5-day washout period during which they used only water on their legs and refrained from using any products other than the test creams. They shaved their legs the day before baseline measurements and did not shave again until the study concluded. The baseline measurements were taken by the Corneometer® and VapoMeter® on Day 1 and then Legs 1-5 were applied to the participants calves (3 sites on the back of each calf) for 14 days (Days 2-15). The Corneometer® and VapoMeter® measurements were repeated on Days 7 and 15. After the measurements on Day 15, panelists did not use any of the test products or other moisturizers and then on Day 18, the final TEWL measurements were taken.

Table 11 shows the TEWL results from the clinical trial. A significant reduction in TEWL (i.e., a negative change from the baseline) indicates that the example product being tested is effective in enhancing the skin's barrier function and reducing water loss, which is especially beneficial for individuals with dry or compromised skin.

The results indicate that Examples 4 and 13, both featuring a lamellar gel network, along with Example 14, exhibited a greater negative change from baseline compared to the other examples. Notably, on Day 18, after a 7-day period without product use, Example 4 showed a substantial reduction in water loss, suggesting a lasting improvement in the skin barrier. Example 13 also maintained its beneficial effects on skin barrier improvement during this period.

While Example 14 demonstrated some effectiveness in reducing TEWL, it is expected to lack the skin feel benefits and salt tolerance associated with the lamellar gel networks present in Examples 4 and 13.

TABLE 11
TEWL Results

| | | LSM Baseline Corrected TEWL (g/Hm²) with | | |
|---|---|---|---|---|
| | | Statistical Homogeneous Groupings (p =< 0.05) | | |
| Leg | | Day 7 | Day 14 | Day 18 |
| 1 | Example 1 (Table 7) | −1.892 B | −2.150 B | −1.892 AB |
| 2 | Example 4 (Table 7) | −1.557 B | −3.041 B | −3.294 B |
| 3 | Example 13 (Table 9) | −1.456 B | −2.415 B | −2.305 B |
| 4 | Example 14 (Table 10) | −1.813 B | −2.608 B | −2.858 B |
| 5 | CeraVe ® Daily Moisturizing | −1.312 AB | −1.588 AB | −1.621 AB |
| | Face and Body Lotion for | | | |
| | Normal to Dry Skin (Table | | | |
| | 3) | | | |
| 6 | No Treatment | 0.349 A | −0.139 A | −0.446 A |

### Combinations:

A. A skin care composition comprising:
   a. an emulsifier comprising an anionic surfactant chosen from an alkyl phosphate monoester, an alkyl phosphate diester, salts thereof, or mixtures thereof; and a non-ionic surfactant;
   b. a fatty amphiphile;
   c. from about 0.1% to about 20%, preferably from about 2% to about 17%, more preferably from about 3% to about 15%, and even more preferably from about 4% to about 13% of a humectant;
   d. from about 0.1% to about 20%, preferably from about 1% to about 15%, more preferably from about 3% to about 12%, and even more preferably from about 5% to about 10% of an emollient;
   e. an aqueous carrier;
   wherein at least a portion, preferably a majority, even more preferably 80% or more of the anionic surfactant, non-ionic surfactant, and fatty amphiphile form a lamellar gel network structure.
B. The skin care composition according to Paragraph A, wherein the composition comprises from about 50% to about 95%, preferably about 52% to about 90%, more preferably about 55% to about 85%, and even more preferably from about 60% to about 80%, by weight of the composition, of the liquid carrier.
C. The skin care composition according to Paragraphs A-B, wherein the emollient comprises at least one liquid emollient comprising a melting point below 40° C., preferably below 35° C., and more preferably below 30° C., according to USP-NF General Chapter <741> “Melting range or temperature” method.
D. The skin care composition according to Paragraph C, wherein the composition comprises from about 0.1% to about 15%, preferably from about 1% to about 9%, more preferably from about 1% to about 8%, and even more preferably from about 3% to about 6% of the liquid emollient.
E. The skin care composition according to Paragraphs A-D, wherein the liquid emollient comprises an oil chosen from an ester, alkane, triglyceride, non-volatile silicones, or mixtures thereof.
F. The skin composition according to Paragraphs A-E, wherein the emollient is selected from Caprylic/Capric Triglyceride, Glyceryl Monostearate, Glyceryl Monooleate, Petrolatum, Isopropyl Isostearate, Coco-Caprylate/Caprate, Hydrolyzed Jojoba Esters, Jojoba Esters, Butyrospermum parkii, dimethicone, Dimethiconol, or mixtures thereof.
G. The skin care composition according to Paragraphs A-F, wherein the composition is formulated without, free of, or substantially free of petroleum-based emollients.
H. The skin care composition according to Paragraphs A-G, wherein the humectant is chosen from propylene glycol, sorbitol, hydroxypropyl sorbitol, erythritol, threitol, pentaerythritol, xylitol, glucitol, mannitol, hexane triol, glycerin, ethoxylated glycerine, propoxylated glycerine, or mixtures thereof. In one aspect, the humectant is propylene glycol, or mixtures thereof.
I. The skin care composition according to Paragraphs A-H, wherein the lamellar gel network structure is present according to cryo-SEM images.
J. The skin care composition according to Paragraphs A-I, wherein the weight ratio of the fatty amphiphile to the anionic and nonionic surfactant is greater than about 2:1, preferably greater than about 4:1 to about 100:1, more preferably greater than about 2:1 to about 40:1, and even more preferably greater than about 2:1 to about 10:1.
K. The skin care composition according to Paragraphs A-J, wherein the composition comprises the lamellar gel network in an amount greater than about 0.1%, preferably from about 1% to about 30%, more preferably from about 2% to about 20%, and even more preferably from about 3% to about 10%, by weight of the skin care composition.
L. The skin care composition according to Paragraphs A-K, wherein the composition comprises from about 0.1% to about 15%, preferably from about 0.5% to about 10%, more preferably from about 1% to about 8%, and even more preferably from about 1.5% to about 7%, by weight of the composition, of the fatty amphiphile.
M. The skin care composition according to Paragraphs A-L, wherein the fatty amphiphile comprises a Hydrophilic-Lipophilic Balance (“HLB”) of 6 or less.
N. The skin care composition according to Paragraphs A-M, wherein the fatty amphiphile comprises a melting point of at least about 27° C., according to USP-NF General Chapter <741> “Melting range or temperature” method.
O. The skin care composition according to Paragraphs A-N, wherein the fatty amphiphile comprises a fatty alcohol comprising a hydrophobic tail group selected from an alkyl group of C12-C70 length.
P. The skin care composition according to Paragraph O, wherein the alkyl group is chosen from lauryl, tridecyl, myristyl, pentadecyl, cetyl, heptadecyl, stearyl, arachidyl, behenyl, undecylenyl, palmitoleyl, oleyl, palmoleyl, linoleyl, linolenyl, arahchidonyl, elaidyl, elaeostearyl, erucyl, isolauryl, isotridecyl, isomyristal, isopentadecyl, petroselinyl, isocetyl, isoheptadecyl, isostearyl, isoarachidyl, isobehnyl, gadoleyl, brassidyl, or mixtures thereof.
Q. The skin care composition according to Paragraph P, wherein the alkyl group is chosen from cetyl, stearyl, behenyl, or mixtures thereof.
R. The skin care composition according to Paragraphs A-Q, wherein the fatty amphiphile comprises a fatty acid and/or an alkoxylated fatty acid.
S. The skin care composition according to Paragraph Q, wherein the fatty acid is chosen from behenic acid, stearic acid, C10-40 hydroxyalkyl acid, C32-36 isoalkyl acid coconut acid, erucic acid, hydroxystearic acid, lauric acid, linoleic acid, myristic acid, oleic acid, palmitic acid, PEG-8 behenate, PEG-5 cocoate, PEG-10 cocoate, PEG-2 laurate, PEG-4 laurate PEG-6 laurate, PEG-8 laurate, PEG-9 laurate, PEG-10 laurate, PEG-7 oleate, PEG-2 stearate, PEG-3 stearate, PEG-4 stearate, PEG-5 stearate, PEG-6 stearate, PEG-7 stearate, PEG-8 stearate, PEG-9 stearate, PEG-10 stearate, polyglyceryl-2-PEG-4 stearate, PPG-2 isostearate, PPG-9 laurate, or mixtures thereof.
T. The skin care composition according to Paragraphs A-S, wherein the composition comprises from about 0.10% to about 4%, preferably from about 0.2% to about 3%, more preferably from about 0.4% to about 1.25%, and even more preferably from about 0.5% to about 1% of the emulsifier.
U. The skin care composition according to Paragraphs A-T, wherein the composition comprises from about 0.05% to about 1.5%, preferably from about 0.1% to about 1%, more preferably from about 0.3% to about 0.6%, and even more preferably from about 0.4% to about 0.5%, of the anionic surfactant.
V. The skin care composition according to Paragraphs A-U, wherein the composition comprises from about 0.1% to about 1%, preferably from about 0.2% to about 0.5%, and more preferably from about 0.3% to about 0.5% of the nonionic surfactant.
W. The skin care composition according to Paragraphs A-V, wherein the non-ionic surfactants comprise a polyoxyethylene alkyl ether comprising a chain length of at least about 5 ethylene oxide units, preferably from about 10 to 200 ethylene oxide units.
X. The skin care composition according to Paragraphs A-W, wherein the non-ionic surfactants are chosen from steareth-100, PEG 100 Stearate, alkyl polyglycerides, esters of alkyl polyglycerides, or mixtures thereof.
Y. The skin care composition according to Paragraphs A-X, wherein the composition further comprises from about 0.05% to about 2%, preferably from about 0.1% to about 1.5%, more preferably from about 0.1% to about 1%, and even more preferably from about 0.25% to about 0.6% polymer.
Z. The skin care composition according to Paragraph Y, wherein the polymer comprises an anionic polymer.
AA. The skin care composition according to Paragraph Y, wherein the polymer is chosen from Caesalpinia spinosa gum/ammonium AMPS Crosspolymer, starch acetate/adipate, alpha-glucan hydroxypropyltrimonium chloride, Cyamopsis tetragonoloba (guar) gum, hydroxyethyl acrylate/sodium acryloyldimethyl taurate copolymer, sodium polyacryloyldimethyl taurate, carboxymethyl chitosan, Sphingomonas ferment extract, acrylamide/sodium acryloyldimethyltaurate copolymer, or mixtures thereof.
BB. The skin care composition according to Paragraph AA, wherein the polymer comprises a Caesalpinia spinosa Gum/Ammonium AMPS Crosspolymer.
CC. The skin care composition according to Paragraphs A-BB, wherein the composition further comprises an additional ingredient chosen from preservatives, chelator, pH adjuster, perfume, dye, pigment, mica, Vitamin B3 compound, panthenol, Vitamin E, hyaluronic acid, collagen, trehalose, Vitamin C, retinyl and derivatives thereof, sunscreen actives, or mixtures thereof.
DD. The skin care composition according to Paragraphs A-CC, wherein the emollient comprises dispersed emollient droplets and wherein 50% or more of the dispersed emollient droplets comprise a diameter of from about 1 to about 10 μm, preferably wherein 50% or more of the dispersed emollient droplets comprise a diameter from about 1 to about 5 μm.
EE. The skin care composition according to Paragraphs A-DD, wherein the emollient comprises dispersed emollient droplets and wherein 90% or more of the dispersed emollient droplets comprise a diameter of less than or equal to 20 μm, preferably wherein 90% or more of the dispersed emollient droplets comprise a diameter of less than or equal to 15 μm.
FF. The skin care composition according to Paragraphs A-EE, wherein the composition comprises a peak viscosity of from about 1500 Pa*s to about 50,000 Pa*s, preferably from about 5000 Pa*s to about 45,000 Pa*s, more preferably from about 7500 Pa*s to about 40,000 Pa*s, and even more preferably from about 15,000 Pa*s to about 40,000 Pa*s, according to the Viscosity Test Method.
GG. The skin care composition according to Paragraphs A-FF, wherein the skin care composition is a body lotion.
HH. The skin care composition according to Paragraphs A-GG, wherein the composition comprises a peak viscosity at 25% dilution of from about 1200 Pa*s to about 35,000 Pa*s, preferably from about 5000 Pa*s to about 25,000 Pa*s, and more preferably from about 10,000 Pa*s to about 22,000 Pa*s, according to the Viscosity Test Method.
II. The skin care composition according to Paragraphs A-HH, wherein the composition comprises a peak viscosity at 50% dilution of from about 1000 Pa*s to about 25,000 Pa*s, preferably from about 5000 Pa*s to about 22,000 Pa*s, and even more preferably from about 10,000 Pa*s to about 20,000 Pa*s, according to the Viscosity Test Method.
JJ. The skin care composition according to Paragraphs A-II, wherein the composition comprises a retention of viscosity at 25% dilution of greater than 46%, preferably greater than 50%, more preferably greater than 52%, and even more preferably greater than 55%, according to the Viscosity Test Method.
KK. The skin care composition according to Paragraphs A-JJ, wherein the composition comprises a retention of viscosity at 50% dilution of greater than 40%, preferably greater than 42%, more preferably greater than 45%, and even more preferably greater than 46%.
LL. The skin care composition according to Paragraphs A-KK, wherein the composition comprises a change in break time of less than 1.2 s, preferably less than 1.1 s, according to the Tack Method.
MM. The skin care composition according to Paragraphs A-LL, wherein the composition comprises a change in break time greater than 0.6 s, preferably greater than 0.7 s, and even more preferably greater than 0.8 s, according to the Tack Method.
NN. The skin care composition according to Paragraphs A-MM, wherein the composition comprises a mean break time of from about 0.01 s to about 0.12 s, preferably from about 0.05 s to about 0.1 s, more preferably from about 0.08 s and to about 0.3 s, and even more preferably from about 0.10 s to about 0.25 s, according to the Tack Method.
OO. The skin care composition according to Paragraphs A-NN, wherein the composition comprises a DSC Onset Transition Temperature of greater than 58° C., preferably greater than 60° C., more preferably greater than 62° C., and even more preferably greater than 63° C., according to the DSC Method.
PP. The skin care composition according to Paragraphs A-GO, wherein the composition comprises a DSC Peak Temperature of greater than 60° C., preferably greater than 63° C., and more preferably greater than 66° C., according to the DSC Method.
QQ. Use of the lamellar gel network according to Paragraphs A-PP, wherein the composition is a body lotion with improved skin feel.
RR. The skin care composition according to Paragraphs A-PP, wherein the skin care composition has a yield stress;
wherein 10 g of the skin care composition is combined with 1.5 mL of a 4.11 M sodium chloride in water solution and mixed thoroughly to form a post-saline composition having a yield stress;
wherein the difference between the yield stress of the skin care composition and the yield stress of the post-saline composition is less than 40%, preferably less than 35%, more preferably less than 30%, even more preferably less than 25%, and even more preferably less than 22%, according to the Viscosity Test Method.
SS. The skin care composition Paragraphs A-PP and RR, wherein the skin care composition has a peak viscosity;
wherein 10 g of the skin care composition is combined with 1.5 mL of a 4.11 M sodium chloride in water solution and mixed thoroughly to form a post-saline composition having a peak viscosity;
wherein the difference between the peak viscosity of the skin care composition and the peak viscosity of the post-saline composition is less than 45%, preferably less than 35%, more preferably less than 30%, even more preferably less than 25%, and even more preferably less than 20%, according to the Viscosity Test Method.
TT. The skin care composition according to Paragraphs A-PP and RR-SS, wherein the skin care composition has a viscosity;
wherein the skin care composition is diluted by 25% with water to form a 25%-diluted composition having a viscosity;
wherein the difference between the viscosity of the skin care composition and the viscosity of the 25%-diluted composition is less than be less than 60%, preferably less than 50%, more preferably less than 45%, according to the Viscosity Test Method.
UU. The skin care composition according to Paragraphs A-PP and RR-TT, wherein the skin care composition has a peak viscosity;
wherein the skin care composition is diluted by 50% with water to form a 50%-diluted composition having a peak viscosity;
wherein the difference between the peak viscosity of the skin care composition and the peak viscosity of the 50%-diluted composition is less than 75%, preferably less than 65%, more preferably less than 60%, and even more preferably less than 55% according to the Viscosity Test Method.
VV. The skin care composition according to Paragraphs A-PP and RR-VV, wherein the skin care composition further comprises at least 1%, preferably at least 2%, more preferably at least 3%, and even more preferably at least 5% a skin care active that contains salt chosen from sodium hyaluronate, sodium salicylate, zinc sulfate, sodium ascorbyl phosphate, sodium citrate, niacinamide, or mixtures thereof.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
a. an emulsifier comprising:
i. an anionic surfactant chosen from an alkyl phosphate monoester, an alkyl phosphate diester, salts thereof, or mixtures thereof; and
ii. a non-ionic surfactant;
b. a fatty amphiphile;
c. from about 0.1% to about 20% of a humectant;
d. from about 0.1% to about 20% of an emollient;
e. an aqueous carrier;
wherein at least a portion of the anionic surfactant, non-ionic surfactant, and fatty amphiphile form a lamellar gel network structure.

2. The skin care composition of claim 1, wherein the skin care composition has a peak viscosity;
wherein the skin care composition is diluted by 25% with water to form a 25%-diluted composition having a peak viscosity;
wherein the difference between the peak viscosity of the skin care composition and the peak viscosity of the 25%-diluted composition is less than be less than 60%.

3. The skin care composition of claim 1, wherein the skin care composition has a peak viscosity;
wherein the skin care composition is diluted by 50% with water to form a 50%-diluted composition having a peak viscosity;
wherein the difference between the peak viscosity of the skin care composition and the peak viscosity of the 50%-diluted composition is less than 75%.

4. The skin care composition of claim 1, wherein the skin care composition has a yield stress;
wherein 10 g of the skin care composition is combined with 1.5 mL of a 4.11 M sodium chloride in water solution and mixed thoroughly to form a post-saline composition having a yield stress;
wherein the difference between the yield stress of the skin care composition and the yield stress of the post-saline composition is less than 40%.

5. The skin care composition of claim 1, wherein the skin care composition has a peak viscosity;
wherein 10 g of the skin care composition is combined with 1.5 mL of a 4.11 M sodium chloride in water solution and mixed thoroughly to form a post-saline composition having a peak viscosity;
wherein the difference between the peak viscosity of the skin care composition and the peak viscosity of the post-saline composition is less than 45%.

6. The skin care composition of claim 1, wherein the skin care composition further comprises at least 1% of a skin care active that contains salt chosen from sodium hyaluronate, sodium salicylate, zinc sulfate, sodium ascorbyl phosphate, sodium citrate, niacinamide, or mixtures thereof.

7. The skin care composition of claim 1, wherein the emollient forms droplets in the skin care composition and wherein at least 50% of the droplets have a diameter of from about 1 μm to about 20 μm.

8. The skin care composition of claim, wherein the emollient comprises dispersed emollient droplets wherein in at least 50% of the droplets have a diameter of from about 1 μm to about 10 μm.

9. The skin care composition of claim 1, wherein the composition comprises from about from about 0.1% to about 15% of the emollient and wherein the emollient comprises at least one liquid emollient comprising a melting point below 40° C.

10. The skin care composition of claim, wherein the emollient is chosen from Caprylic/Capric Triglyceride, Glyceryl Monostearate, Glyceryl Monooleate, Petrolatum, Isopropyl Isostearate, Coco-Caprylate/Caprate, Hydrolyzed Jojoba Esters, Jojoba Esters, Butyrospermum parkii, Dimethicone, Dimethiconol, or mixtures thereof.

11. The skin care composition of claim 1, wherein the humectant is chosen from propylene glycol, sorbitol, hydroxypropyl sorbitol, erythritol, threitol, pentaerythritol, xylitol, glucitol, mannitol, hexane triol, glycerin, ethoxylated glycerine, propoxylated glycerine, or mixtures thereof. In one aspect, the humectant is propylene glycol, or mixtures thereof.

12. The skin care composition of claim 1, wherein the non-ionic surfactants are chosen from steareth-100, PEG 100 Stearate, alkyl polyglycerides, esters of alkyl polyglycerides, or mixtures thereof.

13. The skin care composition according to claim 1, further comprising a polymer chosen from Caesalpinia spinosa gum/ammonium AMPS Crosspolymer, starch acetate/adipate, alpha-glucan hydroxypropyltrimonium chloride, Cyamopsis tetragonoloba (guar) gum, hydroxyethyl acrylate/sodium acryloyldimethyl taurate copolymer, sodium polyacryloyldimethyl taurate, carboxymethyl chitosan, Sphingomonas ferment extract, acrylamide/sodium acryloyldimethyltaurate copolymer, or mixtures thereof.

14. The skin care composition of claim 13, wherein the polymer comprises Caesalpinia spinosa Gum/Ammonium AMPS Crosspolymer.

15. The skin care composition of claim 1, wherein the fatty amphiphile comprises a fatty acid and/or an alkoxylated fatty acid.

16. The skin care composition of claim 1, wherein the composition comprises from about 4% to about 15% of a humectant.

17. The skin care composition of claim 1, wherein the composition comprises a change in break time of less than 1.2 s.

18. The skin care composition of claim 1, wherein the composition comprises the lamellar gel network in an amount from about 2% to about 20%, by weight.

19. The skin care composition according to claim 1, wherein the composition comprises a DSC Onset Transition Temperature of greater than 58° C.
